(19) United States
(12) Patent Application Publication
(10) Pub. No.: US 2012/0171168 A1
(43) Pub. Date: Jul. 5, 2012

(54) METHOD FOR TREATING PANCREATITIS WITH MESENCHYMAL STEM CELLS

(76) Inventors: Sun Uk SONG, Seoul (KR); Don-Haeng Lee, Seoul (KR); Soon-Sun Hong, Gyeonggi-do (KR); Kyung Hee Jung, Gyeonggi-do (KR)

(21) Appl. No.: 12/982,738

(22) Filed: Dec. 30, 2010

(51) Int. Cl. A61K 35/18; A61K 35/12; C12Q 1/02; A61P 7/10; A61P 1/18; A61K 35/28; A61P 29/00

(52) U.S. Cl. 424/93.7; 435/29

(57) ABSTRACT

The present application discloses a method of treating pancreatitis by administering to the patient mesenchymal stem cells that are obtained by manipulating a biological sample of cells, which includes multi-lineage stem cells, progenitor cells, other marrow stromal cells: allowing the sample of cells to settle in a container; transferring supernatant from the container to another container; and isolating cells from the supernatant, which has comparatively lower density in the sample.

### BACKGROUND OF THE INVENTION

1. Field of the Invention

The present invention relates to the field of cell isolation. The present invention also relates to methods of isolating various types of stem cells or progenitor cells. The present invention also relates to a method of treating or alleviating pancreatitis by administering to the patient suffering from the condition the isolated stem cells or progenitor cells.

2. General Background and State of the Art

Bone marrow is known to contain hematopoietic and mesenchymal stem and progenitor cells. Hematopoietic stem cells (HSCs) can generate various types of blood cells [1], and marrow stromal cells (MSCs) or mesenchymal stem cells are capable of differentiating into several different tissues including cartilage, bone and adipose [2,3,4]. MSCs were first found by Friedenstein and his colleagues [5] based on their adherence to cell culture dish. Undifferentiated MSC are fibroblast-like in morphology, self-renewable, and capable of differentiating into mainly connective tissues of the mesoderm origin, namely cartilage, bone, and fat. There are no certain cell surface proteins that specifically and uniquely identify MSCs yet. The diversity of characteristics associated with MSC can be explained by differences in tissue origin, isolation methods and culture conditions between laboratories [2, 6, 7, 8]. Although there is no consistency, MSCs expanded in vitro express CD29, CD44, CD73, CD105, CD106, and CD166 [9], but lacks or are dimly positive for hematopoietic surface markers, such as CD11b, CD14, CD31, CD34, or CD45.

Cell populations with characteristics similar to MSC from different sources including mainly bone marrow, umbilical cord blood, and fatty tissue are known. Although it is difficult to identify whether these cells are distinct cell types due to lack of characteristic markers, they have some different level of surface marker expressions and various differentiation abilities, probably due to their distinct isolation and culture methods. The range of differentiation potential of MSCs is expanding, not only to mesoderm lineages but also to endoderm and/or ectoderm lineages. Therefore, the term “multi-lineage stem or progenitor cell (MLS/PC)” is suggested for these types of stem or progenitor cells capable of differentiating to mesoderm, ectoderm and/or endoderm lineages.

MSCs derived from adult bone marrow offer the potential to open a new strategy in medicine due to its ease of isolation and culture, stability of their phenotype in vitro and low or no allogeneic rejection. In fact, experimental evidence of the hypo-immunogenic nature of MSCs in humans and animals has been accumulating [10]. Currently, clinical applications of adult autologous or allogeneic MSCs have been conducted to treat a variety of diseases, and have generated very promising results [11].

Several protocols have been developed for isolation and expansion of MSCs in culture so far. These methods are based on using density-gradient centrifugation [12], FACs sorting [13,14], specific cell surface antibody [12, 15, 17, 18], selective adhesion to laminin-coated plate [19], Hoechest dye exclusion, and size-sieved culture [24]. Potential disadvantages of these methods in terms of clinical applications are the heterogeneity of cultured cells, high risk of contamination, and/or high cost of production. Therefore, a new protocol to isolate highly homogeneous cell populations with less contamination potential and cost is desired for use in clinical settings.

The present application discloses a new isolation method developed to produce a highly homogeneous population of MLSCs with less contamination potential and cost for clinical applications. This method does not necessarily utilize density-gradient centrifugation, antibody selection, or FACS sorting, but preferably uses mainly natural gravity in a non-coated, collagen or polylysine-coated culture dishes and subfractionation cell culture to separate adherent bone marrow cells according to their cell density. Several distinct highly homogeneous populations of MLSC lines derived from single-cell derived colonies were isolated and expanded with this protocol from human bone marrow. These stem cell lines are self-renewable and capable of differentiating into several different lineages, such as chondrogenic, osteogenic, adipogenic, neurogenic, and hepatogenic lineages.

Acute pancreatitis (AP) has an incidence of approximately 40 cases per 100,000 adults per year. Overall, about 20% of patients with acute pancreatitis have a severe course, and 10 to 30% of those with severe acute pancreatitis die [30]. Despite improvements in intensive care treatment during the past few decades, the death rate for AP has not significantly declined [31]. Intra-acinar cell activation of digestive enzymes such as trypsinogen is thought to be the triggering event of the disease, resulting in interstitial edema, vacuolization, inflammation, and acinar cell death [32]. These pathological changes are also responsible for stimulating the production of inflammatory cytokines such as interleukin (IL)-1β, IL-4, IL-5, IL-6, IL-10, interferon gamma (IFN-γ), and tumor necrosis factor-alpha (TNF-α) from macrophages and lymphocytes. Finally, these cytokines trigger inflammatory cascades which lead to systemic inflammatory response syndrome, multiorgan failure, or death [33]. Despite various experimental and clinical tests of potential drugs, only a few available pharmacological options for treating AP have been used [34].

To this end, new stem cell therapies have raised the possibility of improving AP repair. Mesenchymal stem cells (MSCs) are multipotent and differentiate into a range of cell types and are being tested for their regenerative potential [35]. Another fundamental property of MSCs is their potent immunosuppressive activities that have attracted much attention in the context of novel therapeutic strategies for tissue repair and immunomodulation. Although MSCs can be isolated from adult adipose tissue, fetal and neonatal tissues, in most clinical applications they are isolated from bone marrow (BM) [36]. Recent work has ascribed potent tissue regeneration, repair, and anti-inflammatory effects to BM-derived MSCs in various inflammation-based diseases such as kidney disease in ischemia/reperfusion injury, collagen-induced arthritis, and acute renal failure [37-39].

So far, most preclinical and clinical studies have used a mixed population of mononuclear cells. Previous studies have reported that mature MSCs are large, while rapidly self-renewing cells are small [40]. Therefore, several protocols have been developed for isolation and expansion of MSCs with high efficiency. The isolation of MSCs by using the conventional gradient centrifugation method produces a heterogeneous population, which has led to confused results in the outcomes of clinical trials [41]. In any case, at least a standardized isolation procedure is necessary to obtain a homogenous population of MSCs for human clinical trials. We have recently developed a new protocol for the isolation of a homogeneous population of MSCs called the subtraction culturing method (SCM), and established a library of human clonal MSC (hcMSC) lines [42].

Although the physiological and protective effects of MSCs for various other diseases [37-39] have been reported, the effects of MSCs on AP have not been reported yet. In the current study, we used hcMSCs that have been obtained via SCM and investigated whether hcMSCs can recover impaired pancreas by migrating to injured pancreas and have anti-inflammatory effects in mammals with both mild- and severe AP according to grade severity.

hcMSCs that have been obtained via subfractionation culturing method are used to determine whether impaired pancreas can recover by the migration of hcMSCs to injured pancreas and causing anti-inflammatory effects in rats with both mild- and severe AP according to grade severity.

### SUMMARY OF THE INVENTION

The invention provides adult stem or progenitor cells that can be used to treat pancreatitis.

In one aspect, the invention is directed to a method of treating pancreatitis, or reducing pancreatic edema or reducing relative weight of pancreas or increasing population of acinar cells or preventing necrosis of acinar cells of a subject suffering from pancreatitis, which may include the steps of:

(i) obtaining a biological sample of bone marrow, peripheral blood, cord blood, fatty tissue sample, or cytokine-activated peripheral blood cells;

(ii) allowing the biological sample of bone marrow, peripheral blood, cord blood, fatty tissue sample, or cytokine-activated peripheral blood cells to settle in a container;

(iii) transferring supernatant which contains comparatively less dense cells in the sample compared to other cells from the container to another container in a serial manner at least two times;

(iv) isolating lower dense cells from the supernatant; and

(v) administering the cells obtained in step (iv) to a subject suffering from pancreatitis, wherein the bone marrow, peripheral blood, cord blood, fatty tissue sample, or cytokine-activated peripheral blood cells do not undergo centrifugation of greater than 1,000 rpm in steps (i) to (iii), so as not to disturb the integrity of the cells or cause dissociation of cells from the tissue.

The sample of cells may be mixed with a growth medium that does not contain any enzyme that causes dissociation of cells from the tissue, such as a protease. In a preferred embodiment, the steps (ii) and (iii) may be carried out at least three times. The isolated cells from the supernatant may be expanded in a container. The container may have a flat bottom. The container is coated with a cell adhesive agent. The cell adhesive agent may include a polymer of any charged amino acids. The cell adhesive agent may be collagen, polylysine, polyarginine, polyaspartate, polyglutamate, or a combination thereof. The sample of cells may be obtained from bone marrow. A single colony of multi-lineage stem cells or progenitor cells may be isolated. The biological sample of cells may be obtained prior to undergoing any centrifugation. The biological sample of cells may be obtained after undergoing any centrifugation. The method may exclude centrifugation of the sample of cells. The method may not use specific antibody detection of cells. Pancreatitis may be mild acute pancreatitis. The pancreatitis may be severe acute pancreatitis. The subject may be treated without systemic immunosuppression.

In one aspect, firstly, the subject suffering from pancreatitis may be identified or diagnosed as a subject with the conditions that satisfy the criteria of pancreatitis. Further, upon treatment of the subject with the isolated multi-lineage stem cells using the method described in the present application, the subject may be tested to determine whether the pancreatitis has been treated or alleviated using methods known in the art or methods disclosed in the present application.

The subject may be tested for the effectiveness of the treatment by comparing reduction of pancreatic edema or reduction of relative weight of pancreas or increase in population of acinar cells or lessening of necrosis of acinar cells in the subject, before and after treatment, or relative to healthy control, which may include healthy control pancreas.

In one aspect, the invention is concerned with isolating multi-lineage stem cells. The cells may be progenitor cells.

Yet in another embodiment, the biological sample of cells may be obtained after undergoing centrifugation, preferably mononuclear cells isolated or fractionated by conventional density-gradient centrifugation method typically employed for MSC isolation. In another embodiment, the cell isolation method may exclude any enzyme in the isolation media such that the separation of cells is caused by the difference in density between the cells.

1. In an alternative aspect, the invention is directed to a method of treating pancreatitis, or reducing pancreatic edema or reducing relative weight of pancreas of a subject suffering from pancreatitis, which includes the following steps;

(A) obtaining a homogeneous population of single cell-derived clonal multipotent bone marrow cells based on cell density from a biological sample, comprising:

(i) allowing the biological sample to settle by gravity in a first container producing a first supernatant of lower density cells;

(ii) transferring the first supernatant directly without undergoing centrifugation to a second container of growth medium and allowing cells to settle to the bottom producing a second supernatant of lower density cells;

(iii) transferring the second supernatant directly without undergoing centrifugation to a third container of growth medium and allowing cells to settle to the bottom, producing a third supernatant of lower density cells;

(iv) transferring the third supernatant directly without undergoing centrifugation to another container of growth medium and allowing cells to settle to the bottom, producing another supernatant of lower density cells;

(v) allowing single-cell derived colonies to appear on the bottom of any of the container of growth medium to which supernatant is transferred;

(vi) isolating the single-cell derived colonies; and

(vii) expanding cells from the single-cell derived colonies in a further other container of growth medium to obtain the homogeneous population of single cell-derived clonal multipotent bone marrow cells; and

(B) administering the cells obtained in (A) to a subject suffering from pancreatitis.

2. The method according to 1 above, wherein the cells are settled for one day in step (iv).

3. The method according to 1 above, wherein the isolated cells from the supernatant are expanded in a container.

4. The method according to 1 above, wherein the container has a flat bottom.

5. The method according to 1 above, wherein the container is coated with a cell adhesive agent.

6. The method according to 5 above, wherein the cell adhesive agent comprises a polymer of any charged amino acids.

7. The method according to 6 above, wherein the cell adhesive agent is collagen, polylysine, polyarginine, polyaspartate, polyglutamate, or a combination thereof.

8. The method according to 1 above, which does not use specific antibody detection of cells.

9. In another aspect, the invention is directed to a method of treating pancreatitis, or reducing pancreatic edema or reducing relative weight of pancreas of a subject suffering from pancreatitis, which includes the following steps;

(A) obtaining a homogeneous population of single cell-derived clonal multipotent bone marrow cells based on cell density from a biological sample, comprising:

(i) allowing the biological sample to settle by gravity in a first container producing a first supernatant of lower density cells;

(ii) transferring the first supernatant directly without undergoing centrifugation to a second container of growth medium and allowing cells to settle to the bottom producing a second supernatant of lower density cells;

(iii) transferring the second supernatant directly without undergoing centrifugation to a third container of growth medium and allowing cells to settle to the bottom, producing a third supernatant of lower density cells;

(iv) transferring the third supernatant directly without undergoing centrifugation to a fourth container of growth medium and allowing cells to settle to the bottom, producing a fourth supernatant of lower density cells;

(v) transferring the fourth supernatant directly without undergoing centrifugation to another container of growth medium and allowing cells to settle to the bottom, producing another supernatant of lower density cells;

(vi) allowing single-cell derived colonies to appear on the bottom of any of the container of growth medium to which supernatant is transferred;

(vii) isolating the single-cell derived colonies; and

(viii) expanding cells from the single-cell derived colonies in a further other container of growth medium to obtain the homogeneous population of single cell-derived clonal multipotent bone marrow cells; and

(B) administering the cells obtained in (A) to a subject suffering from pancreatitis.

10. The method according to 9 above, wherein the cells are settled for one day in step (v).

11. The method according to 9 above, wherein the isolated cells from the supernatant are expanded in a container.

12. The method according to 9 above, wherein the container has a flat bottom.

13. The method according to 9 above, wherein the container is coated with a cell adhesive agent.

14. The method according to 13 above, wherein the cell adhesive agent comprises a polymer of any charged amino acids.

15. The method according to 14 above, wherein the cell adhesive agent is collagen, polylysine, polyarginine, polyaspartate, polyglutamate, or a combination thereof.

16. The method according to 9 above, which does not use specific antibody detection of cells.

17. In yet another aspect, the invention is directed to a method of treating pancreatitis, or reducing pancreatic edema or reducing relative weight of pancreas of a subject suffering from pancreatitis, which includes the following steps;

(A) obtaining a homogeneous population of single cell-derived clonal multipotent bone marrow cells based on cell density from a biological sample, comprising:

(i) allowing the biological sample to settle by gravity in a first container producing a first supernatant of lower density cells;

(ii) transferring the first supernatant directly without undergoing centrifugation to a second container of growth medium and allowing cells to settle to the bottom producing a second supernatant of lower density cells;

(iii) transferring the second supernatant directly without undergoing centrifugation to a third container of growth medium and allowing cells to settle to the bottom, producing a third supernatant of lower density cells;

(iv) transferring the third supernatant directly without undergoing centrifugation to a fourth container of growth medium and allowing cells to settle to the bottom, producing a fourth supernatant of lower density cells;

(v) transferring the fourth supernatant directly without undergoing centrifugation to a fifth container of growth medium and allowing cells to settle to the bottom, producing a fifth supernatant of lower density cells;

(vi) transferring the fifth supernatant directly without undergoing centrifugation to another container of growth medium and allowing cells to settle to the bottom, producing another supernatant of lower density cells;

(vii) allowing single-cell derived colonies to appear on the bottom of any of the container of growth medium to which supernatant is transferred;

(viii) isolating the single-cell derived colonies; and

(ix) expanding cells from the single-cell derived colonies in a further other container of growth medium to obtain the homogeneous population of single cell-derived clonal multipotent bone marrow cells; and

(B) administering the cells obtained in (A) to a subject suffering from pancreatitis.

18. The method according to 17 above, wherein the cells are settled for one day in step (vi).

19. The method according to 17 above, wherein the isolated cells from the supernatant are expanded in a container.

20. The method according to 17 above, wherein the container has a flat bottom.

21. The method according to 17 above, wherein the container is coated with a cell adhesive agent.

22. The method according to 21 above, wherein the cell adhesive agent comprises a polymer of any charged amino acids.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows overall flow diagram for the isolation of multi-lineage stem cells from human bone marrow using a subfractionation culturing method. In brief, 1 ml of human bone marrow was mixed with 15 ml of DMEM-HG, DMEM-LG, or a-MEM (20% FBS) and plated onto 10 cm² cell culture dish. After 2 hour incubation, only supernatant was transferred to a new dish. This was repeated once more. The supernatant was then transferred to a non-coated, collagen- or polylysine-coated dish. From this stage, the cells were incubated for 1 day twice and 2 days once. The final supernatant was incubated until single clones of cells appeared. When single clones of cells were big enough to transfer to 6-well plate, the cells were expanded to larger plates for further studies.

FIGS. 2A-2D show the morphological characteristics of isolated multi-lineage stem cells from bone marrow. (A & B) MLSCs three days after the final subfractionation of bone marrow cells. Cells have fibroblast-like morphology. Magnification: (A) 40× and (B) 200×. (C) Cells reached confluence with a consistent and homogeneous morphology at day seven. (D) After six passages of the isolated MLSCs, the morphology of a small portion (less than 2 to 3%) of MLSCs was changed to a wider and larger shape, compared to the ones at earlier passages. The morphology of the isolated and expanded MLSCs is spindle shape which is similar to known marrow stromal stem cells.

FIGS. 3A-3D show the morphology of four established multi-lineage stem cell lines from bone marrow. Pictures of four established multi-lineage stem cell lines, called A. D4(#1), B. D4(#3), C. D5(#1), and D. D5(#2), grown to about 70 to 80% confluence. The morphology of the established multi-lineage stem cell lines is spindle shaped and these stem cells grow as fast as other fibroblast cells.

FIG. 4 shows the cell surface proteins of isolated MLSCs from bone marrow by a subfractionation culturing method. Flow cytometry analyses showed that MLSCs were consistently positive for typical MSC integrin protein (CD29) and matrix receptors (CD44 and CD105). HMSC8292 (Cambrex Bio Science, Walkersville, Md., USA) cells were used as a control. The cell surface proteins which are known to be expressed for typical MSC are also expressed in MLSCs, suggesting that MLSCs could have MSC characteristics.

FIG. 5 shows no hematopoietic stem cell surface proteins are observed on isolated MLSCs from bone marrow by a subfractionation culturing method. Flow cytometry analyses showed that MLSCs were negative for HLA-DR and CD34 marker proteins for early hematopoietic stem cells. HMSC8292 (Cambrex Bio Science, Walkersville, Md., USA) cells were used as a control. These results indicate that the isolated MLSCs do not have hematopoietic stem cell phenotypes.

FIG. 6 shows comparison of cell surface protein CD31 (PECAM) expression observed on isolated MLSC lines from bone marrow by a subfractionation culturing method. Expression of CD31 of D4(#1), D4(#3), D5(#1), D5(#2), and D5(#2) with FGF were measured by FACS analysis. The established MLSC line D4(#3) is dimly positive for CD 31, whereas the other MLSC lines are negative. FGF in the growth medium increases the expression of CD31 of D5(#2). These results indicate that D4(#3) has different cell characteristics in differentiation capability and/or cell function.

FIG. 7 shows comparison of cell surface protein CD105 (SH2) expression observed on isolated MLSC lines from bone marrow by a subfractionation culturing method. Expression of CD105 of D4(#1), D4(#3), D5(#1), D5(#2), and D5(#2) with FGF were measured by FACS analysis. The established MLSC line D5(#1) shows an intermediate level of CD105 expression, whereas the other stem cell lines show high level of CD105. These results suggest that D5(#1) has different cell characteristics in differentiation capability and/or cell function.

FIG. 8 shows comparison of cell surface protein CD73 (SH3, SH4) expression observed on isolated MLSC lines from bone marrow by a subfractionation culturing method. Expression of CD73 of D4(#1), D4(#3), D5(#1), D5(#2), and D5(#2) with FGF were measured by FACS analysis. The established MLSC line D4(#1) shows a very low level of CD 73 expression and D4(#3) and D5(#2) show an intermediate level, whereas D5(#1) does not express it at all. These results suggest that each stem cell lines has unique cell characteristics in its differentiation capability and/or cell function.

FIG. 9 shows comparison of cell surface protein CD34 expression observed on isolated MLSC lines from bone marrow by a subfractionation culturing method. Expression of CD34 of D4(#1), D4(#3), D5(#1), D5(#2), and D5(#2) with FGF were measured by FACS analysis. The established MLSC lines D4(#3), D4(#3), and D5(#2) show low level of CD34 expression, whereas D5(#1) shows no CD34 expression. These results indicate that each stem cell line has unique cell characteristics in its differentiation capability and/or cell function.

FIGS. 10A-10D show chondrogenic differentiation of the isolated MLSCs. Histochemical stain with Toluidine-blue showed that chondrogenically differentiated MLSCs were highly positive for the stain, tested 21 days after chondrogenic induction. (A & B) Cell pellet grown in regular medium. (C & D) Cell pellet grown in chondrogenic induction medium. The results show that MLSCs grown in chodrogenic induction medium secrete high level of proteoglycans and can be differentiated into chodrocytes.

FIGS. 11A-11D show. Osteogenic differentiation of the isolated MLSCs. Histochemical stain with von Kossa stain showed the presence of mineral associated with the matrix in the osteogenically differentiated MLSCs, 21 days after osteogenic induction. (A & B) Cell pellet grown in regular medium. (C & D) Cell pellet grown osteogenic induction medium. The results show that MLSCs grown in osteogenic induction medium can make high level of calcium and can be differentiated into osteocytes.

FIGS. 12A-12D. Adipogenic differentiation of the isolated MLSCs. Histochemical stain with Oil red-O showed that adipogenically differentiated MLSCs were highly positive for the stain, tested 21 days after adipogenic induction. (A & B) Cell pellet grown in regular medium. (C & D) Cell pellet grown adipogenic induction medium. The results show that MLSCs grown in adipogenic induction medium can produce neutral lipid vacuoles and can be differentiated into adipocytes.

FIGS. 13A-13I show neurogenic differentiation of the isolated MLSCs. Immunohistological stain with GFAP, Nestin, and NeuN antibodies showed that neurogenically differentiated MLSCs were highly positive for the stain, tested 7 and 14 days after neurogenic induction. (A, D & G) MLSCs grown in normal culture medium and incubated with the antibodies. (B, E & H) Cells stained with each antibody 7 days after neurogenic induction. (C, F & I) Cells stained with each antibody 14 days after neurogenic induction. The results show that MLSCs grown in neurogenic induction medium can synthesize glial cell specific protein, glial fibrillary acidic protein (GFAP), early and late neural cell marker proteins, Nestin and NeuN, respectively and can be differentiated into neural cells.

FIGS. 14A-14I show neurogenic differentiation of the isolated MLSCs grown with FGF. Immunohistological stain with GFAP, Nestin, and NeuN antibodies showed that neurogenically differentiated MLSCs grown with FGF were highly positive for the stain, tested 7 and 14 days after neurogenic induction. (A, D & G) MLSCs grown in normal culture medium and incubated with the antibodies. (B, E & H) Cells stained with each antibody 7 days after neurogenic induction. (C, F & I) Cells stained with each antibody 14 days after neurogenic induction. The results show that MLSCs grown in neurogenic induction medium with FGF can also synthesize glial cell specific protein (GFAP), early and late neural cell marker proteins, Nestin and NeuN, respectively and can be differentiated into neural cells.

FIGS. 15A-15D shows morphological changes of the isolated MLSCs grown in hepatogenic induction medium. Morphological changes were observed 14 days after growing in hepatogenic induction medium. (A) Morphology of MLSCs grown in normal culture medium. (B, C & D) Hepatological morphology changes of MLSCs grown in hepatogenic induction medium for 14 days. The results show that MLSCs grown in hepatogenic induction medium can be differentiated into hepatocytes.

FIGS. 16A-16E show observation of chondrocyte, osteocyte, adipocyte, hepatocyte, and neural cell specific gene expression by RT-PCR analysis. Total RNA was analyzed by RT-PCR for the expression of (A) type II collagen (chondrogenic, 500 bp), (B) osteopontin (osteogenic, 330 bp), (C) peroxisome proliferator activated receptor gamma 2 (PPARγ2) (adipogenic, 352 bp), (D) neurofilament molecule (NF-M) (neurogenic, 430 bp), and (E) alpha feto protein (αFP) (hepatogenic, 216 bp). Expression of Glyceraldehyde-3-phosphate dehydrogenase (GAPDH) was used as an internal control. M; DNA molecular size markers, N: non-induced, C: chondrogenic, O: osteogenic, A: adipogenic, Ne: neurogenic, and H: hepatogenic. These results strongly indicate that the isolated MLSCs can express cell-specific genes in each specific differentiation condition and can be differentiated into multi-lineages.

FIG. 17 shows cytokine secretion of isolated MLSC lines. Aliquots (50˜100 μl) of the MLSC culture supernatant were analyzed by ELISA using the Quantikine® Human TGF-β1, b-NGF, LIF, IL10, HGF, IL2, TGF-α and IL12. TGF-β1, LIF, TGF-α, and IL10 showed high levels of secretion, whereas the others showed low or no secretion. High level of TGF-β1 secretion by the isolated MLSCs indicates that these stem cells can play a role in suppression of T-cell activation. Also, relatively high level of other cytokines, such as LIF, TGF-α, and IL10, suggest that these cells may have immune-modulation activities.

FIGS. 18A-18C show effect of hcMSCs on different concentration (×10⁵ and ×10⁶) in rats with mild-AP. (A) Pancreas sections of CM-DiI-labeled hcMSCs at 1×10⁵ and 1×10⁶, injected into rats with mild-AP (B) Histological analysis of AP. (C) Pancreas/body-weight ratio and activities of amylase (U/L), lipase (U/L), and myeloperoxidase (U/mL). Con, control; Con+hcMSCs, hcMSCs alone-infusion group; AP, mild-AP group; AP+hcMSCs, hcMSCs infusion in mild-AP group. Original magnification ×200.

FIGS. 19A-19C show characterization of hcMSCs isolated from human bone marrow. (A) Fibroblast-like shapes of hcMSCs on plastic culture dishes and crystal violet staining for clear visualization of hcMSCs. (B) Expression of several stem cell markers by using flow cytometry. (C) The multilineage potential of hcMSCs, adipogenic, osteogenic, and chondrogenic differentiation and molecular markers of gene expression during each differentiation by RT-PCR. Original magnification ×40 and 100.

FIGS. 20A-20C show effects of hcMSCs in rats with mild- and severe-AP. (A) Histological analysis of AP. (B) Decreased apoptotic cells by hcMSCs in mild- and severe-AP. (C) Pancreas/body-weight ratio and activities of amylase (U/L), lipase (U/L), and myeloperoxidase (U/mL). Each value represents the mean±SD of four separate experiments. *P<0.05 and **P<0.01, compared to mild- and severe-AP group. Con, control; Con+hcMSCs, hcMSCs alone-infusion group; AP, mild- and severe-AP group; AP+hcMSCs, hcMSCs infusion in mild- and severe-AP group. Original magnification ×200.

FIGS. 21A-21C show tracking of infused hcMSCs. (A) Pancreas sections of CM-Dil-labeled hcMSCs at 1×10⁶, injected into rats with or without mild- or severe-AP. (B) human genomic AluI PCR from pancreas tissues. Sample 1, Con; sample 2 and 3, Con+hcMSCs; sample 4 and 5, mild-AP+hcMSCs; sample 6 and 7; severe-AP+hcMSCs, sample 8, human DNA. (C) Distribution of hcMSCs in various tissues. Distribution was assessed from pancreas, lung, liver, spleen, and kidney sections after CM-DiI-labeled hcMSCs (1×10⁶) injection in rats with or without mild- or severe-AP. The numbers of CM-DiI-labeled hcMSCs represents as mean±SD at least ten fields. Con+hcMSCs, hcMSCs alone-infusion group; mild- and severe-AP+hcMSCs, hcMSCs infusion in mild- and severe-AP group. Original magnification ×200.

FIG. 22 shows combined FISH for human chromosome and immunofluorescent staining. A representative image from combined FISH for a human-specific chromosome after CM-DiI labeled hcMSCs in rats with mild- and severe-AP. Mild- and severe-AP+hcMSCs, hcMSCs infusion in mild- and severe-AP; hCEN, human chromosome centromere. Original magnification ×200.

FIGS. 23A-23B show inflammatory cytokines and mediators after infusion of hcMSCs. (A) mRNA expression levels. (B) TGF-β, TNF-α, and IFN-γ serum levels after hcMSCs infusion by enzyme-linked immunosorbent assay. Data are the mean±SD for at least three separate experiments. *P<0.05 and **P<0.01, compared to mild- and severe-AP. Con, control; Con+hcMSCs, hcMSCs alone-infusion group; AP, mild- and severe-AP; AP+hcMSCs, hcMSCs infusion in mild- and severe-AP.

FIGS. 24A-24E show suppression of T cells by hcMSCs. (A) PBMC and lymph node cells were isolated from two different rats and human bloods, respectively. S: SD, W: Wistar, A, B: PBMC donors, M: hcMSCs, L: lymphocytes. (B) Lymph node cells from rats were stimulated with indicated stimuli and Foxp3⁺ and Annexin V (C) expression were measured in CD4⁺ T cells. (D) Inhibition of T-cell infiltration and (E) expression of Foxp3⁺ by hcMSCs in pancreas tissues. Data represents as mean±SD for at least five fields. *P<0.05 and **P<0.01, compared to mild- and severe-AP. Con, control; Con+hcMSCs, hcMSCs alone-infusion group; AP, mild- and severe-AP; AP+hcMSCs, hcMSCs infusion in mild- and severe-AP. Original magnification ×200.

FIG. 25 shows a table of FISH analysis of hcMSCs in rat pancreas after hcMSCs infusion.

### DETAILED DESCRIPTION OF THE PREFERRED EMBODIMENTS

In the present application, “a” and “an” are used to refer to both single and a plurality of objects.

Occasionally, abbreviations are used to describe the present invention. As used herein, the following abbreviations shall have the following meanings: hcMSCs, human clonal bone marrow-derived mesenchymal stem cells; CM-DiI, CM-1,1′-dioctadecyl-3,3,3′-tetramethylindo-carbocyanine perchloride; AP, acute pancreatitis; IFN, interferon, IL, interleukin; MPO, myeloperoxidase; TGF, transforming growth factor; iNOS, inducible nitric oxide synthase; TNF, tumor necrosis factor.

As used herein, “bodily sample” refers to any sample obtained from a mammal from which is desired to isolate a single type of cell. Such bodily sample includes bone marrow sample, peripheral blood, cord blood, fatty tissue sample, and cytokine-activated peripheral blood.

As used herein, “mammal” or “subject” for purposes of discussing the source of the cells and treatment refers to any animal classified as a mammal, including humans, domestic and farm animals, and zoo, sports, or pet animals, such as dogs, cats, cattle, horses, sheep, pigs, rats, mice, rabbits, and so on. Preferably, the mammal is human.

As used herein, “sample of cells” refers to any sample in which is contained a mixture of different types of cells, including bone marrow sample, peripheral blood, cord blood, fatty tissue sample, and cytokine-activated peripheral blood.

As used herein, “homogeneous” population of cells generally indicates that the same type of cells are present within the population. Substantially homogeneous may mean about 80% homogeneity, or about 85%, 90%, 91%, 92%, 93%, 94%, 95%, 96%, 97%, 98%, 99%. 99.1%, 99.2%, 99.3%, 99.4%, 99.5%, 99.6%, 99.7%, 99.8%, or 99.9% homogeneity.

As used herein, “lower density cell” refers to cells that have lower density than others in the sample, and are the object of isolation. The lower density cell includes without limitation, multi-lineage stem cells, progenitor cells, other marrow stromal cells.

As used herein, “MLSC” refers to multi-lineage stem cell.

As used herein, “MLSC/PC” refers to multi-lineage stem cell or progenitor cell.

As used herein, “MSC” refers to marrow stromal cells or mesenchymal stem cells, which terms are used interchangeably.

Subfractionation Technique

The present application describes a method, named subfractionation culturing method, that isolates a highly homogeneous population of multi-lineage stem cells (MLSCs) from a bodily sample or source such as human bone marrow. A total of sixteen bone marrow cell lines were established out of one ml of bone marrow aspirate. Of the sixteen, four cell lines showing distinct phenotypes by FACS analysis were further characterized. All of these cell lines showed characteristics of multi-lineage stem cells, such as the self-renewal ability and the capacity of differentiating into mesoderm, ectoderm, and endoderm lineage cells.

Bone marrow MSCs have been known to be difficult to isolate without contamination by hematopoietic cells [20, 21]. For application in clinical settings, it is important to have a homogeneous population of MSCs in order to prevent immunogenic problems and to evaluate the clinical effects correctly. Conventionally, isolation of homogeneous populations of MSCs was carried out by MSC-specific antibody column purification. However, even this method is not adequate as no such perfect MSC-specific antibody is available yet.

A rationale for the inventive method for isolating MLSCs from a biological sample such as a bone marrow sample is that multi-lineage stem or progenitor cells have low cell density and therefore they can be separated from other cells in the sample on this basis. For example, mature MSCs are larger than rapidly self-renewing (RS) cells [22, 23]. RS cells are known to possess a greater capacity for multi-lineage differentiation.

In another aspect, collagen or polylysine-coated culture dishes were used in order to obtain more adherent stem cells. Applicant has discovered that any charged culture surface, either positive or negative, helps the attachment of stem cells to it, compared to the surface of a non-coated dish. More cells were attached to a collagen or polylysine-coated culture dish than a non-coated dish, approximately about two to three fold respectively (data not shown). Similar results were obtained with other human bone marrow aspirates and three different strains of mouse bone marrow samples in terms of obtaining of single-cell derived marrow cell colonies (data not shown), indicating that this protocol is consistent with other bone marrow aspirates and can be applied to isolate MLSCs in other species as well.

Thus, in one embodiment, the bottom of a culture dish can be coated by either positively charged amino acids, such as polylysine, polyarginine, or negatively charged amino acids, such as polyaspartate, polyglutamate, or a combination thereof to help stem or progenitor cells adhere better to the bottom of the dish.

To practice the inventive subfractionation culturing method, it is not necessary to employ centrifugation of any type to pre-remove any type of cells such as red or white blood cells from the sample because most of the heavier or more dense cells can be removed within the first two, 2-hour incubation steps. In this regard, it is also not necessary to pre-treat the cells with any enzymes to digest away any material between the cells. Thus, one advantage of the inventive system is that conventionally used gradient centrifugation and mononuclear cell fractionation steps, which may introduce contamination such as Picoll, Ficoll or Ficoll-hypaque into the cell culture may be avoided. Accordingly, the inventive subfractionation culturing method is a simple, effective, and economic protocol to isolate highly homogeneous MLSCs from a bodily sample, preferably a bone marrow sample.

Alternatively, mononuclear cells isolated/fractionated by conventional density-gradient centrifugation method of MSC isolation can also be subjected into the D1 dish to obtain single cell-derived colonies and then to isolate homogeneous populations of stem or progenitor cells. Therefore the fractionation culturing method can be used with the mononuclear cells fractionated by the conventional density-gradient centrifugation.

The present application describes diversity of characteristics in cell surface protein expression of the isolated single-cell derived stem cell lines, which indicates that there are several different types of multi-lineage stem or progenitor cells that exist in biological samples, and in particular bone marrow samples, which are exemplified. The isolated MLSCs were generally negative or dimly positive for CD34, HLA-DR, CD73, CD31, CD166, HLA Class I and highly positive for CD44, CD29, CD105. However, some cell lines from D4 and D5 dishes exhibited distinctive levels of surface proteins, which indicates that there could be several different types of multi-lineage stem or progenitor cells in bone marrow. Hung et al. also speculated that bone marrow may include many groups of MSCs that are different in surface marker analyses [24]. These MSCs having different surface markers may represent different differentiation potential of the cells. Therefore, isolation of single-cell derived homogeneous stem cells by the inventive subfractionation culturing method makes it possible to isolate tissue-specific stem or committed progenitor cells, as long as these groups of cells exist in the bone marrow or other specifically isolated bodily sample, and culture conditions do not change their potential during cell expansion. The safety and efficacy of MSC treatment and cell engraftment process is improved by being able to characterize subpopulations of cells with specific properties, as shown in the present application.

The present application shows a novel method that isolates a highly homogeneous population of MLSC lines derived from single cells from any other bodily sample, bone marrow cells in particular, with the capacity of renewal and multi-lineage differentiation into ectoderm, mesoderm, and endoderm lineage cells. By eliminating density-gradient centrifugation and mononuclear cell fractionation steps and without requiring the use of antibodies to separate stem cells, the inventive subfractionation culturing method generates more homogeneous populations of MLSCs in a simple, effective, and economic procedure and safer applications for therapeutic settings.

Induction, Differentiation/Transformation Agents for Endoderm Cell Lineage

Induction, differentiation/transformation agents for endoderm cell lineage include the following agents: hepatocyte growth factor, oncostatin-M, epidermal growth factor, fibroblast growth factor-4, basic-fibroblast growth factor, insulin, transferrin, selenius acid, BSA, linoleic acid, ascorbate 2-phosphate, VEGF, and dexamethasone, for the following cell types: liver, lung, pancreas, thyroid, and intestine cells.

Induction, Differentiation/Transformation Agents for Mesoderm Cell Lineage

Induction, differentiation/transformation agents for mesoderm cell lineage include the following agents: insulin, transferrin, selenous acid, BSA, linoleic acid, TGF-β1, TGF-β3, ascorbate 2-phosphate, dexamethasone, β-glycerophosphate, ascorbate 2-phosphate, BMP, and indomethacine, for the following cell types: cartilage, bone, adipose, muscle, and blood cells.

Induction, Differentiation/Transformation Agents for Ectoderm Cell Lineage

Induction, differentiation/transformation agents for ectoderm cell lineage include the following agents: dibutyryl cyclin AMP, isobutyl methylxanthine, human epidermal growth factor, basic fibroblast growth factor, fibroblast growth factor-8, brain-derived neurotrophic factor, and/or other neurotrophic growth factor, for the following cell types: neural, skin, brain, and eye cells.

Mild and Severe Acute Pancreatitis

Acute pancreatitis (AP) is an acute inflammatory condition of the pancreas that may extend to local and distant extrapancreatic tissues. AP is broadly classified as mild or severe.

Mild AP, called interstitial or edematous pancreatitis is characterized by minimal or no organ dysfunction and by a prompt uncomplicated recovery. Overall, about 80% of patients with AP have the mild disease [89]. The predominant macroscopic feature is interstitial edema. Lipolysis of the intra- and peripancreatic adipose tissue is commonly seen [90].

Severe AP implies the presence of organ failure, local complications, or pancreatic necrosis. The significant histopathological findings of AP are interstitial edema, necrosis, inflammation, and hemorrhage. These alterations occur as a result of the lytic pancreatic enzyme liberation into the pancreatic interstitium and damage of the glandular parenchyma, blood vessels, and fat tissue [91].

AP according to the severity of the edema, inflammation, necrosis, and hemorrhage can be identified as edematous (mild-AP) and hemorrhagic/necrotizing pancreatitis (Severe-AP).

The pathogenesis of these pancreatitis types are the same, but the pathological and clinical findings are dependent on the degree of the damage. Also, clinically, patients with AP are classified as having severe AP if they suffer at least one of the following criteria: (1) organ failure (systolic arterial pressure below 90 mmHg (=shock), paO2=60 mmHg or less (=respiratory insufficiency), serum creatinin >2 mg/dL after rehydration (=renal failure), and evidence of digestive hemorrhage at a rate above 500 mL/day); (2) evidence of local complications (necrosis, infection, presence of an abscess, or a pseudocyst); (3) a Ranson score greater than or equal to 3, and/or (4) an APACHE II score greater than or equal to 8 [92-95]. A Ranson score cannot be completed before 48 h after admission and APACHE II can be calculated after 24 h.

Treatment of Pancreatitis

Pancreatitis is inflammation of the pancreas that can occur in two very different forms. Acute pancreatitis is sudden while chronic pancreatitis is characterized by recurring or persistent abdominal pain with or without steatorrhea or diabetes mellitus.

Types of pancreatitis includes acute pancreatitis, chronic pancreatitis and pancreatic abscess. Acute pancreatitis is swelling (inflammation) of the pancreas. The most common cause of acute pancreatitis is the presence of gallstones—small, pebble-like substances made of hardened bile—that cause inflammation in the pancreas as they pass through the common bile duct. Excessive alcohol use is the most common cause of chronic pancreatitis, and can also be a contributing factor in acute pancreatitis.

Less common causes include, hypertriglyceridemia (but not hypercholesterolemia) and only when triglyceride values exceed 1500 mg/dl (16 mmol/L); hypercalcemia; viral infection (e.g., mumps); trauma (to the abdomen or elsewhere in the body) including post-ERCP (i.e., Endoscopic Retrograde Cholangiopancreatography); vasculitis (i.e., inflammation of the small blood vessels within the pancreas), and autoimmune pancreatitis. In addition, pancreas divisum, a common congenital malformation of the pancreas may underlie some cases of recurrent pancreatitis.

Chronic pancreatitis is inflammation of the pancreas that leads to scarring and loss of function. This makes the pancreas unable to produce the right amount of enzymes needed to digest fat. It also interferes with insulin production, which may lead to diabetes. The condition is most often caused by alcoholism and alcohol abuse.

A pancreatic abscess is a cavity of pus within the pancreas. Pancreatic abscesses develop in patients with pancreatic pseudocysts that become infected. Patients with pancreatic abscesses usually have had pancreatitis.

To date, it has not been possible to prevent AP or to arrest the disease process completely through medical treatment. In this study, we evaluated the value of hcMSCs for the improvement of AP as cell-based therapeutic strategy. The major findings in our study were as follows: (1) hcMSCs, obtained from our new protocol, had multipotential and immunosuppressive properties in vitro. (2) Administration of the hcMSCs alleviated not only mild-AP but also severe-AP, showing pancreatic edema, infiltration of inflammation cells, and necrosis of acinar cells. (3) After infusion of CM-DiI labeled hcMSCs to rats, hcMSCs were more detected in acutely injured pancreas than normal pancreas. Interestingly, more hcMSCs was observed in severe-AP than mild-AP, and infused hcMSCs were observed to recombine with human chromosome by FISH analysis in rat pancreas. (4) hcMSCs recovered pancreas function by decreasing the expression of inflammatory mediators/cytokines and inhibiting T-cell infiltration as well as up-regulating expression of Foxp3⁺ regulatory T cell. It is believed that this is the first report showing that hcMSCs improved mild- and severe-pancreatic injury induced by cerulein and TCA, inhibiting inflammatory responses in animal models with AP.

AP is a frequent gastrointestinal disorder with an unpredictable clinical course that as yet has no satisfactory therapy [48]. MSCs infusion could be beneficial for the repair of tissue injury but also on that MSCs have immunosuppressive properties [49], which permits them to be used in allogenic or xenogenic conditions. In this study, we isolated and characterized a new population of MSCs from human BM, termed hcMSCs. We demonstrated that hcMSCs exhibited in vitro multipotent differentiation into different cell lineages including adipogenic, osteogenic, and chondrogenic cells, and expression of MSCs surface markers. In addition, hcMSCs seemed to suppress both rat and human T-cell proliferation. These findings show that hcMSCs, obtained via this new protocol, SCM, possess self-renewal, differentiation capacity, and xenogenic T-cell suppression capacity.

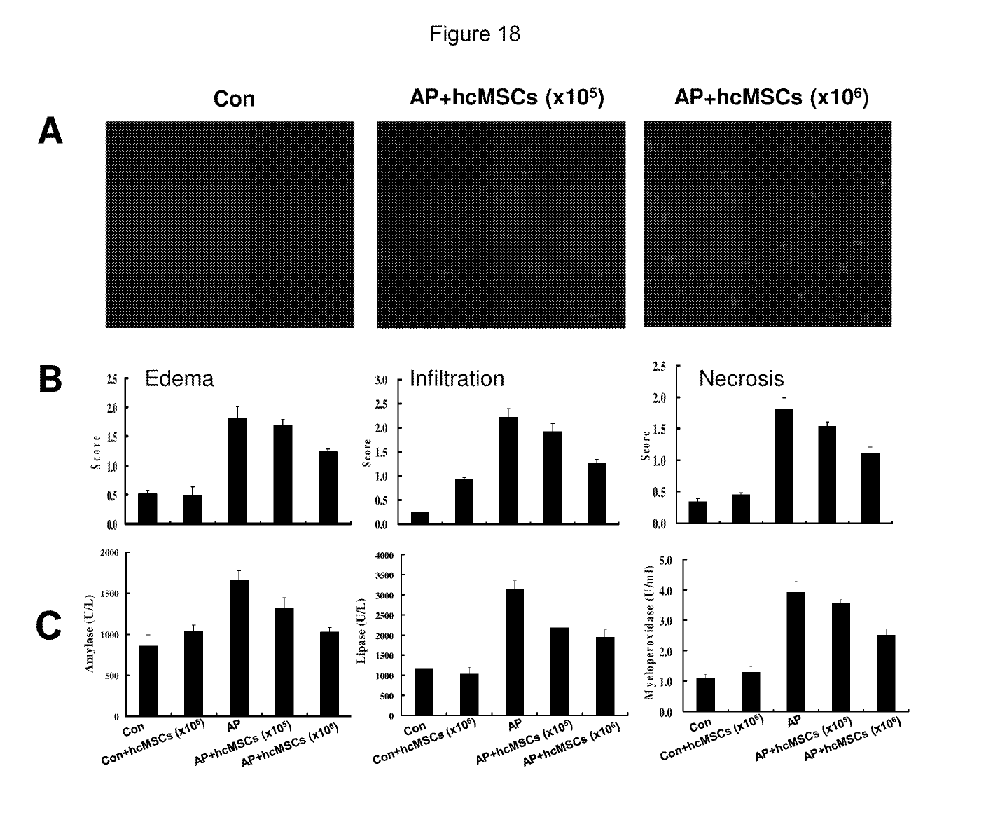

For MSC therapy to work, it is crucial that MSCs reach the site of injury. Some studies have indicated that systemically delivered MSCs can indeed migrate to the kidney, lung, liver, and hind limb after its injury [50-53]. To assess the contribution of hcMSCs to pancreatic tissue, we used two hcMSCs cell track markers: CM-DiI and human-specific chromosome centromere by FISH. The fluorescent dye CM-DiI has been used in several studies to track implanted stem cells after pre-labeling [54, 55]. However, the presence of rare, scattered autofluorescent cells does limit the specificity of the marker, and thus it is important to confirm our findings using in situ hybridization, a more specific technique. In our study, we observed that considerable numbers of CM-DiI-labeled hcMSCs migrated into the site of inflammation of the pancreas and recombined with human-specific chromosome centromere in pancreas of mild- and severe-AP rats, showing that infused hcMSCs were well incorporated into the injured pancreas. Surprisingly, more hcMSCs was detected in severe-AP than in mild-AP. This indicates that the tissue injury may play important roles in migration of hcMSCs to pancreas and action of hcMSCs are likely to differ depending on degree of injury, period of hcMSCs infusion, and cell number of hcMSCs (FIGS. 18A-18C). Distribution data of hcMSCs into other tissues showed that much lower number of hcMSCs was observed in lung, liver, spleen, and kidney compared with the injured pancreas. Our results suggest that hcMSCs preferentially migrate to injured pancreas without the need for systemic immunosuppression and retain in the organ, colonize there and contribute to healing of the AP.

Because of their ability to migrate to sites of tissue injury, MSCs have been used as a promising therapeutic modality for tissue repair in acute diseases. MSC infusion has shown beneficial effects on several models of acute injury, such as acute lung injury, myocardial infarction, graft-versus-host disease and kidney injury [56-58]. However, very few studies to date have investigated the potential role of cell therapy for pancreatitis. Previous studies have focused on pancreatic islets [59, 60]. Thus, we investigated the effects of hcMSCs on mild- and severe-AP. In our two AP models, hcMSCs significantly ameliorated pancreas function, as detected by reduction of pancreatic edema and relative pancreatic weight, and mitigation of all the histological alterations. We also confirmed that hcMSCs reduced the elevation of amylase, lipase, and MPO. These results show that hcMSCs are potentially capable of limiting pancreatic damage produced during AP by restoring the fine structure of acinar cells. Recently, Marrache et al. [61] reported that BM-derived progenitor cells contributed to pancreatic tissue repair in experimental chronic pancreatitis [61]. However, there are at least two major distinctions between Marrache et al. and the present invention, in terms of cells and model type. Marrache et al. conducted syngeneic administration using progenitor cells obtained from mouse BM in chronic pancreatitis, whereas the present application discloses xenogeneic administration with clonal MSCs of human BM in AP. Based on these results, hcMSCs have the greater possibility of clinical use.

Inflammation plays an important role in the pathology of AP. The manifestations of the disease are mediated by different pro- and anti-inflammatory cytokines released during the course of pancreatitis [62]. TNF-α, IL-1β, and IL-6 as proinflammatory cytokines are mainly produced during AP [63, 64]. Studies of Masamune et al. [65] and Cuzzocrea et al. [66] reported that anti-cytokine therapies against TNF-α, IL-1β, and IL-6 showed protective effect in experimental animal models of AP [65, 66]. Also, the study of Ishibashi et al. [67] reported that severe-AP increased the level of TNF-α and IL-6 [67]. In addition, IFN-γ is presumed to be involved in various kinds of inflammatory diseases [68, 69]. Indeed, Hayashi et al. observed that mild-AP augmented the intrapancreatic expression of both IFN-γ mRNA and protein, along with pancreatic tissue damage and massive neutrophil infiltration [88]. In addition, enhanced productions of TGF-β and iNOS are involved in the pathogenesis of human and experimental pancreatitis [70-72]. In contrast to pro-inflammatory cytokines, IL-4 and IL-10 block inflammation as major anti-inflammatory cytokines [73, 74]. The information available regarding IL-4 and IL-10 in AP is controversial, and different studies show a great variability in the results obtained [75-79]. Elevated levels of IL-4 and IL-10 have been documented in human and animal models with pancreatitis [75, 80, 81], whereas Laveda et al. and Zhang et al. reported that IL-4 and IL-10 in AP were at low levels and the administration of exogenous IL-4 and IL-10 protected the pancreas against acute damage [76, 77]. In this study, we observed that hcMSCs decreased the expression of proinflammatory cytokines such as TNF-α, IL-1β, IL-6 and other inflammatory cytokines and mediators in parallel with decreasing anti-inflammatory cytokines in both mild- and severe-AP. Zhang et al. and Guo et al. reported similar results, as demonstrated by the fact that human gingiva-derived MSCs attenuated the levels of TNF-α, IL-1β, IL-6, IFN-γ, and IL-17 in myocardial infarction and colitis [82, 83]. In addition, our results are in line with those of Semedo et al., who reported that MSCs increased the levels of IL-4 and IL-10 after acute kidney injury [84].

In this regard, the decrease of inflammatory cytokines and mediators, and increase of anti-inflammatory cytokines by hcMSCs may explain their association with pathological and functional improvements in AP. Recent studies focused that the capacity of MSCs to regulate various inflammatory mediators may suppress the immune response by Foxp3⁺ regulatory T cells [45, 85]. Also, Foxp3⁺ regulatory T cells reported to induce apoptosis of neutrophil and CD4⁺ T cells [86, 87]. Therefore, we identified whether hcMSCs give rise to induction of expression of Foxp3⁺ regulatory T cells and apoptosis of T cells. We revealed that hcMSCs can induce regulatory T cell generation and suppress T cell proliferation via apoptosis which result in the decreased T cell infiltration in hcMSCs-treated pancreatic tissue.

In conclusion, we have shown that hcMSCs have a capacity for xenogenic T-cell suppression in rats. Most importantly, hcMSCs are capable of improving pancreatic damage and exert anti-inflammatory effects through induction of Foxp3⁺ regulatory T cell generation and suppression of T cell proliferation at least in rats with mild- and severe-AP.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Isolation of MLSCs and Cell Culture

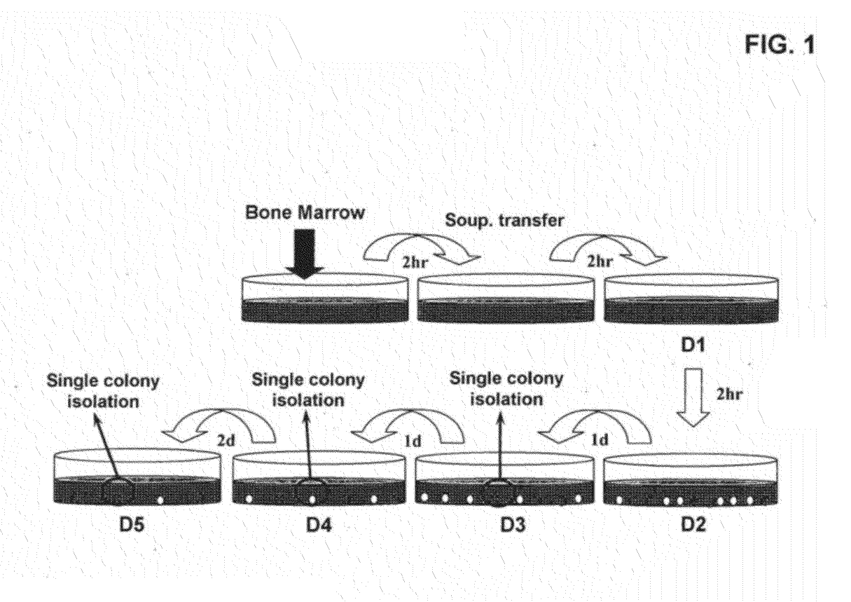

One-ml of discarded human bone marrow aspirate, taken from the iliac crest of a patient undergoing bone marrow examination after informed consent and approval of the Inha University Medical School IRB, were mixed with 15 ml of complete growth medium: Dulbecco's modified Eagle's Medium (DMEM) containing high or low glucose (GIBCO-BRL, Life-technologies, MD USA), with 20% fetal bovine serum (FBS) and 1% penicillin/streptomycin, and then incubated in 100 mm culture dish. As shown in FIG. 1, after 2 hour incubation at 37° C., 5% CO₂, only the cell culture supernatant was transferred to a new dish. After another 2 hour incubation in a new dish, the supernatant was transferred to a non-coated, collagen or polylysine-coated dish and incubated for 2 hours (D1). After transferring the supernatant one more time to a new dish (D2), the supernatant was transferred to a new dish and then incubated for 1 day (D3). This was repeated two more times with 1 and 2 day incubation (D4 and D5 respectively). The single colonies grown in the D4 or D5 dish were transferred to a 100 mm plate first and then kept expanded in larger culture flasks. After usually 10 to 14 days in the 100 mm plate, the cells were harvested with 0.25% trypsin and 1 mM EDTA (GIBCO-BRL), suspended at 1×10⁶ cells/ml in 10% dimethylsulfoxide (DMSO) and 40% FBS, and frozen in 1 ml aliquots in liquid nitrogen (passage 1). For detaching and isolating of single colonies, trypsin/EDTA was used for 1-2 minutes with a sterile cylinder. Once the cells reached about 80 to 90% confluence, they were recovered with trypsin/EDTA and replated at 50-100 cells/cm².

### Example 2

### Flow Cytometry Analysis

The isolated and expanded cells from single-cell derived colonies were characterized at passage 3 or 4 by flow cytometric analysis for a panel of cell surface proteins. The cells were harvested from 75 flask by treatment of trypsin/EDTA and washed with PBS twice. The cells were incubated in PBS with 0.1% goat serum for blocking and then washed with washing buffer (PBS with 0.4% BSA) twice. The cells were incubated with fluorescein isothiocyanate (FITC) or phycoerythrin (PE)-conjugated antibodies for 40 min at 4° C. Tested antigens included matrix receptors (CD13, CD44, CD105), integrin (CD29), PECAM (CD31), ALCAM (CD166), SH3 and SH4 (CD73), Thy-1 (CD90) and hematopoietic lineage markers (CD34, HLA-DR, HLA-ClassI) (BD Biosciences Pharmingen, San Diego, Calif., USA). The cell mixture was then washed twice with washing buffer and analyzed using a fluorescence-activated cell sorter (FACS) with a 525 nm filter for green FITC fluorescence and with a 575 nm filter for red PE fluorescence. As a control, human mesenchymal stem cells (HMSC 8292, Cambrex Bio Science, Walkersville, Md. USA) were used.

### Example 3

### Induction of Multi-Lineage Differentiation

Pellet culture assay was used for chondrogenic, osteogenic, adipogenic differentiation experiments using passage 3 or 4 cells. 2×10⁵ cells in 0.5 ml culture medium were spun down to make a pellet. The following supplements in DMEM containing high glucose and 20% FBS were used for each lineage, chondrogenic differentiation medium: 6.25/ml insulin (Sigma Chemical Co, St Louis, Mo., USA), transferrin (Sigma), 6.25 ng/ml selenous acid (Sigma), 1.25 mg/ml BSA (Sigma), 5.35/ml linoleic acid (Sigma), TGF-β1 10 ng/ml (R&D Systems, Minneapolis, Minn., USA), and TGF-β3 10 ng/ml (R&D Systems, Minneapolis, Minn., USA); osteogenic differentiation medium: 50/ml ascorbate 2-phosphate (Sigma), 10⁻⁸ M dexamethasone (Sigma), and 10 mM β-glycerophosphate (Sigma); adipogenic differentiation medium: 50/ml ascorbate 2-phosphate (Sigma), 10⁻⁷ M dexamethasone (Sigma), and 50/ml indomethacine (Sigma). The pellet culture was incubated at 37° C., 5% CO₂ and medium was changed every 3 days.

For neurogenic differentiation, the isolated and expanded cells at passage 3 were seeded into a 6 well culture plate at a concentration of 1×10⁴ cells with basic medium. After 24 hours, the basic medium was discarded and replaced by neuronal differentiation medium. The cells were cultured 1 mM dibutyryl cyclin AMP (dbcAMP; Sigma, St. Louis, Mo.), 0.5 mM isobutyl methylxanthine (IBMX; Sigma, St. Louis, Mo.), 20 ng/ml human epidermal growth factor (hEGF; Sigma, St. Louis, Mo.), 40 ng/ml basic fibroblast growth factor (bFGF; Sigma, St. Louis, Mo.), 10 ng/ml fibroblast growth factor-8 (FGF-8; PEPROTECH INC, Rocky Hill, N.J.), 10 ng/ml brain-derived neurotrophic factor (BDNF; R&D Systems, Minneapolis, Minn.). NEUROBASAL™ media (GIBCO BRL, Gaithersburg, Md.) with 1×B27 supplement (GIBCO BRL, Gaithersburg, Md.) is a serum-free basal medium for the long-term viability of hippocampal and other neurons of the central nervous systems.

For hepatocyte differentiation, the isolated and expanded cells at passage 4 were plated at a concentration 1×10⁴ cells into 60 mm dish. After 24 hours, the cells were treated with differentiation medium containing 20 mg/ml hepatocyte growth factor (R&D), 10 ng/ml oncostatin-M (R&D), 10 ng/ml epidermal growth factor (sigma), 20 ng/ml fibroblast growth factor-4 (R&D), 10 ng/ml basic-fibroblast growth factor (sigma), 50 mg/ml ITS+ premix (Becton Dickinson; 6.25 ug/ml insulin, 6.25 ug/ml transferrin, 6.25 ng/ml selenius acid, 1.25 mg/ml BSA, 5.35 mg/ml linoleic acid)), 0.1 μM ascorbate 2-phosphate (sigma), 10⁻⁸M dexamethasone (sigma). Medium was changed every 3 days.

### Example 4

### Histochemical and Immunohistochemical Staining

Histochemical staining and immunohistochemistry study were performed 14 or 21 days after the initiation of differentiation culture. The pellets were washed with PBS twice after removing the differentiation medium. The pellets were embedded with OCT compound (Sakura Finetek, Torrance, Calif., USA) and 6 sections were stained. The tissues were stained with toluidine blue, von Kossa, and Oil red-O to show chondrogenic, osteogenic, and adipogenic differentiation respectively. Immunohistochemical staining for human type II collagen was also performed to demonstrate chondrogenic differentiation of the tissue.

For the immunocytochemical staining of neuronal cells, all wells were then fixed with 99.9% ethanol and labeled with mouse anti-neuronal nuclear antigen (NeuN, 10 ug/ml) IgG monoclonal antibody (Chemicon, Temecula, Calif.), mouse anti-nestin (5 ug/ml) IgG monoclonal antibody (Chemicon, Temecula, Calif.) and monoclonal anti-Glial Fibrillary Acidic Protein (GFAP, 1:400; Sigma, St. Louis, Mo.) for 1 hour at room temperature. The cells were then rinsed with PBS, and immunostaining was detected using the Histostain-Plus Kit (Zymed Laboratories Inc., San Francisco, Calif.). DAB served as the chromogen. Cells were photographed with a digital camera to assess the positive expression of neuronal specific markers.

### Example 5

### RNA Extraction and RT-PCR Analysis

Total RNA was extracted from the non-differentiated and differentiated cells using TRIZOL® (Invitrogen Co, Carlsbad, Calif., USA) reagent. Complementary DNA was synthesized with total RNA (1) using Reverse Transcription System Kit (Promega). PCR was performed using specific primers designed for each lineage as follows: col-2 (500 bp), sense: 5′-AAGATGGTCCCAAAGGTGCTCG-3′ (SS101-F SEQ ID NO:1) and antisense: 5′-AGCTTCTCCTCTGTCTCCTTGC-3′ (SS101-R SEQ ID NO:2); osteopontin (330 bp), sense: 5′-CTAGGCATCACCTGTGCCATACC-3′ (SS102-F SEQ ID NO:3) and antisense: 5′-CGTGACCAGTTCATCAGATTCATC-3′ (SS102-R SEQ ID NO:4), PPAR-γ2 (352 bp), sense: 5′-GCTGTTATGGGTGAAACTCTG-3′ (SS103-F SEQ ID NO:5) and antisense: 5′-ATAAGGTGGAGATGCAGGCTC-3′ (SS103-R SEQ ID NO:6), GAPDH (350 bp), sense: 5′-AACTCCCTCAAGATTGTCAGCA-3′ (SS104-F SEQ ID NO:7) and antisense: 5′-TCCACCACCCTGTTGCTTGTA-3′ (SS104-R SEQ ID NO:8), NF-M (430 bp), sense: 5′-GAG CGCAAAGACTACCTGAAGA-3′ (SS105-F SEQ ID NO:9) and antisense: 5′-CAGCGATTTCTATATCCAGAGCC-3′ (SS105-R SEQ ID NO:10), and αFP (216 bp), sense: 5′-TGCAGCCAAAGTGAAGAGGGAAGA-3′ (SS106-F SEQ ID NO:11) and antisense: 5′-CATAGCGAGCAGCCCAAAGAAGAA-3′ (SS106-R SEQ ID NO:12). PCR was performed for 35 cycles with each cycle of denaturing at 95° C. for 1 min, annealing at 56° C. for 1 min, and elongating at 72° C. for 1 min. The amplified DNA products were run on a 1% agarose gel.

### Example 6

### Results

### Example 6.1

### Isolation and Expansion of Bone Marrow Cell Colonies

In order to explore if it is possible to isolate human bone marrow stem or progenitor cells by subfractionation culturing method, as described in FIG. 1, bone marrow was mixed with culture medium and kept fractionated by transferring only the cell culture supernatant to new dishes. The rationale of this fractionation is based on the hypothesis that bone marrow stem or progenitor cells may have low cell density. It was usually not possible to obtain well-separated single colonies in D1 and D2 dishes. There were at least few different types of cells observed with distinct morphology and size in D1 and D2 dishes, indicating the cellular heterogeneity in marrow-derived adherent monolayer cultures. The adherent cells in D1 or D2 culture dish reached confluence at 7 to 10 or 14 to 21 days respectively after transferring cell culture supernatant from the previous dish. It became possible to obtain well-separated single-cell derived colonies in D3, D4, and D5 dishes. The initial adherent spindle-shaped cells appeared as single colonies between 14 to 21 days after transferring culture supernatant from the previous dish. Ten, three, and three single-cell derived colonies appeared in D3, D4, or D5 dish respectively.

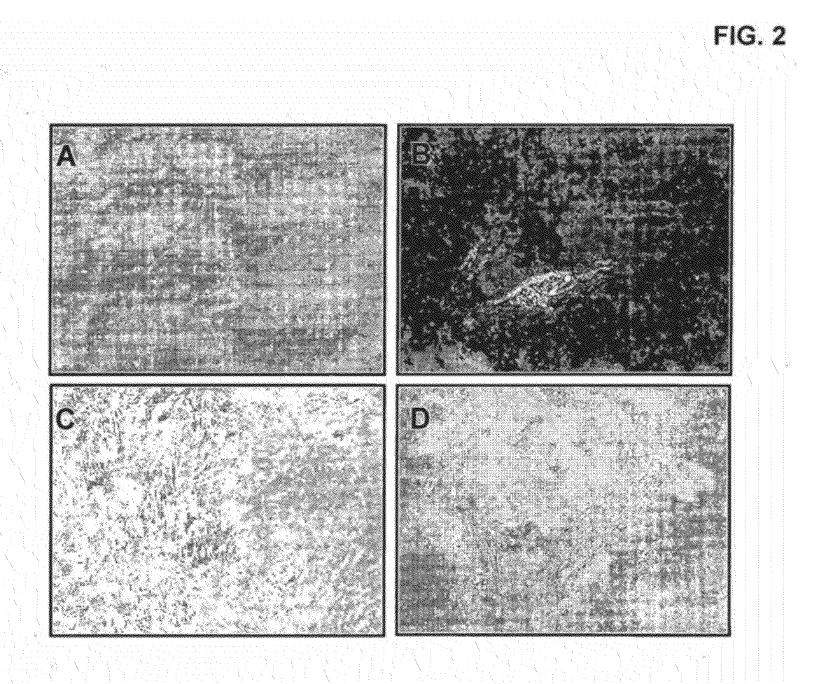

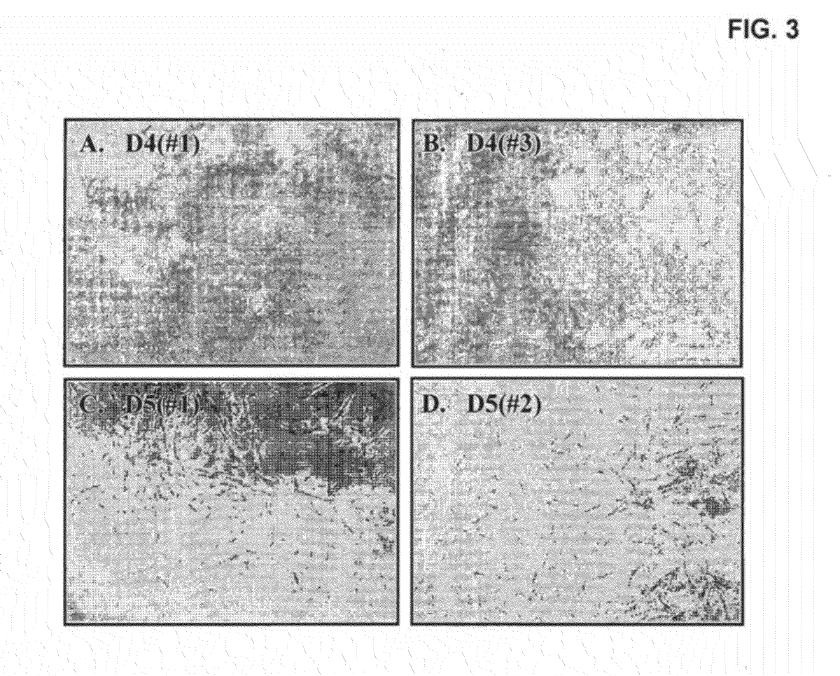

FIG. 2 shows the morphological characteristics of isolated multi-lineage stem cells from bone marrow three days after the final subfractionation of bone marrow cells. The cells have fibroblast-like morphology. The cells reached confluence with a consistent and homogeneous morphology at day seven. After six passages of the isolated MLSCs, the morphology of a small portion (less than 2 to 3%) of MLSCs was changed to a wider and larger shape, compared to the ones at earlier passages. The morphology of the isolated and expanded MLSCs is spindle shape which is similar to known marrow stromal stem cells. Once the colonies of approximately 200 to 300 cells were formed, the cells proliferated rapidly as fast as normal fibroblast cells do. Among the six cell lines generated from the individual colonies in D4 and D5 dishes, four cell lines showed distinct phenotypes by FACS analysis and were further characterized. These cell lines at 70-80% confluence in culture dishes are shown in FIG. 3.

### Example 6.2

### Phenotypic Characterization of Bone Marrow Cell Lines

To characterize the phenotypes of single-cell derived bone marrow cell lines, a panel of cell surface proteins was analyzed by FACS analysis, as summarized in Table 1.

TABLE 1
Summary of cell surface protein expression of the
isolated MLSC lines assayed by FACS analysis

| Cell Surface | | | | | |
|---|---|---|---|---|---|
| Protein | D4 (#1) | D4 (#3) | D5 (#1) | D5 (#2) | D5 (#2, FGF) |
| CD 13 | L | L | N | L | L |
| CD 29 | H | H | I | H | H |
| CD 31 | L | L | N | N | I |
| CD 34 | L | L | N | L | L |
| CD 44 | H | H | H | H | H |
| CD 73 | L | L | N | L | I |
| CD 90 | H | H | H | H | I |
| CD 105 | H | H | I | H | H |
| CD 166 | H | H | I | H | H |
| HLA-DR | N | L | N | N | L |
| HLA-ClassI | H | H | I | H | H |

(N—negative, L—low, I—intermediate, H—high)

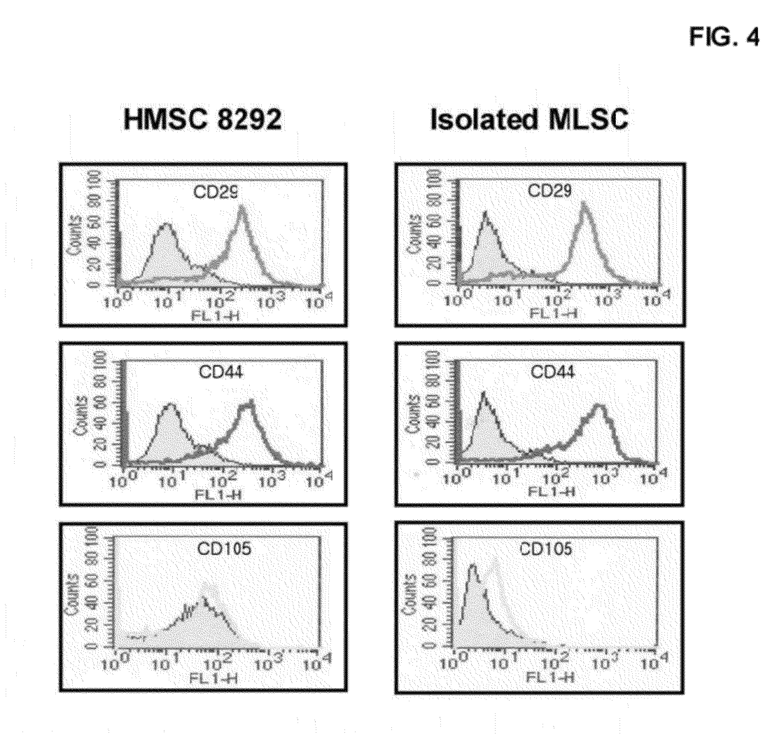

The results showed that overall profiles of surface expression are similar, for example, all the isolated cell lines were strongly positive for CD29, CD44, CD73 (SH3, SH4), CD90, CD105 (SH2), CD166, and HLA-ClassI. However, among the 11 cell surface proteins tested, each stem cell line has relatively unique expression profiles in 9 cell surface protein expressions and similar level of expressions in CD44 and CD90. Further, D5 (#1) cell line was negative for CD31, CD34 and HLA-DR and D5 (#2) was CD31, HLA-DR negative but CD34 was dimly positive, whereas D5 (#3) was positive for CD31, CD34 and HLA-DR (FIG. 4). These results indicate that several different types of stem cells exist in human bone marrow.

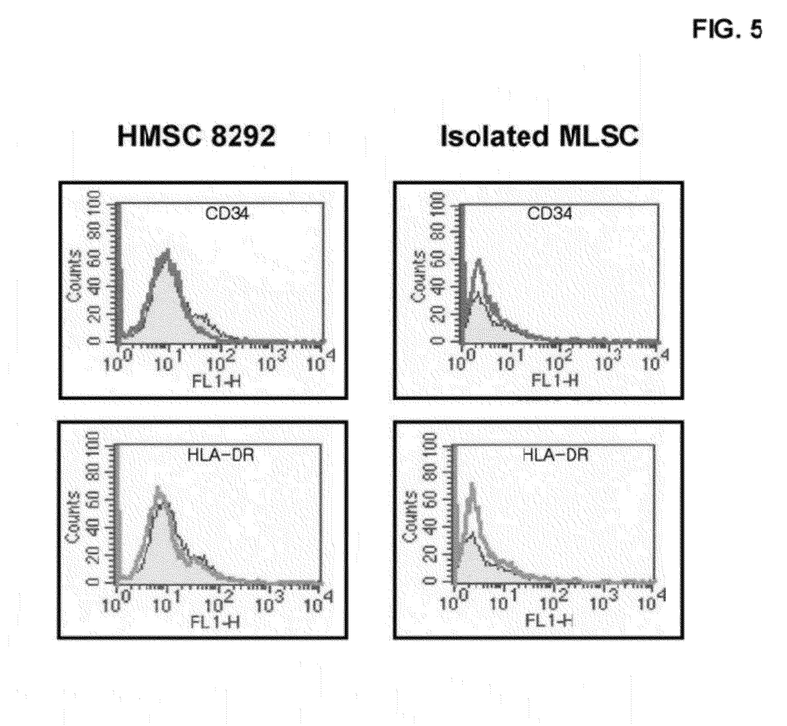

FIG. 5 shows that there are no hematopoietic stem cell surface proteins observed on isolated MLSCs from bone marrow by the inventive subfractionation culturing method. Flow cytometry analyses showed that MLSCs were negative for HLA-DR and CD34 marker proteins for early hematopoietic stem cells. HMSC8292 (Cambrex Bio Science, Walkersville, Md., USA) cells were used as a control. These results indicate that the isolated MLSCs do not have hematopoietic stem cell phenotypes.

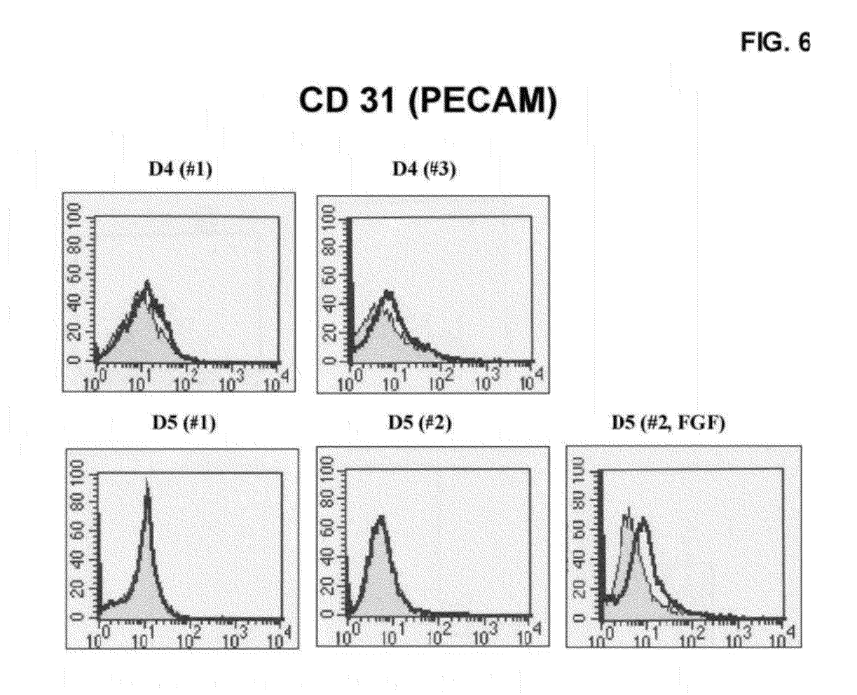

With respect to the expression of several representative surface proteins markers CD31, CD105, CD73, and CD34 on the isolated cell lineages, FIGS. 6-9 show their comparisons. FIG. 6 shows a comparison of cell surface protein CD31 (PECAM) expression observed on isolated MLSC lines from bone marrow by the inventive subfractionation culturing method. Expression of CD31 of D4(#1), D4(#3), D5(#1), D5(#2), and D5(#2) with FGF were measured by FACS analysis. The established MLSC line D4(#3) is dimly positive for CD 31, whereas the other MLSC lines are negative. FGF in the growth medium increases the expression of CD31 of D5(#2). These results indicate that D4(#3) has different cell characteristics in differentiation capability and/or cell function.

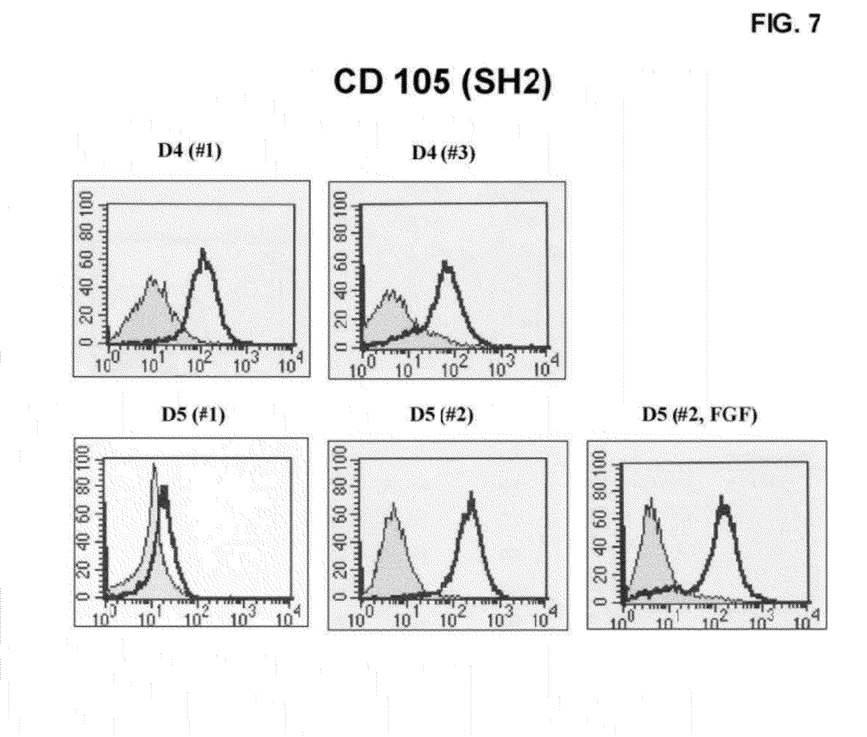

FIG. 7 shows a comparison of cell surface protein CD105 (SH2) expression. The established MLSC line D5(#1) shows an intermediate level of CD105 expression, whereas the other stem cell lines show high level of CD105.

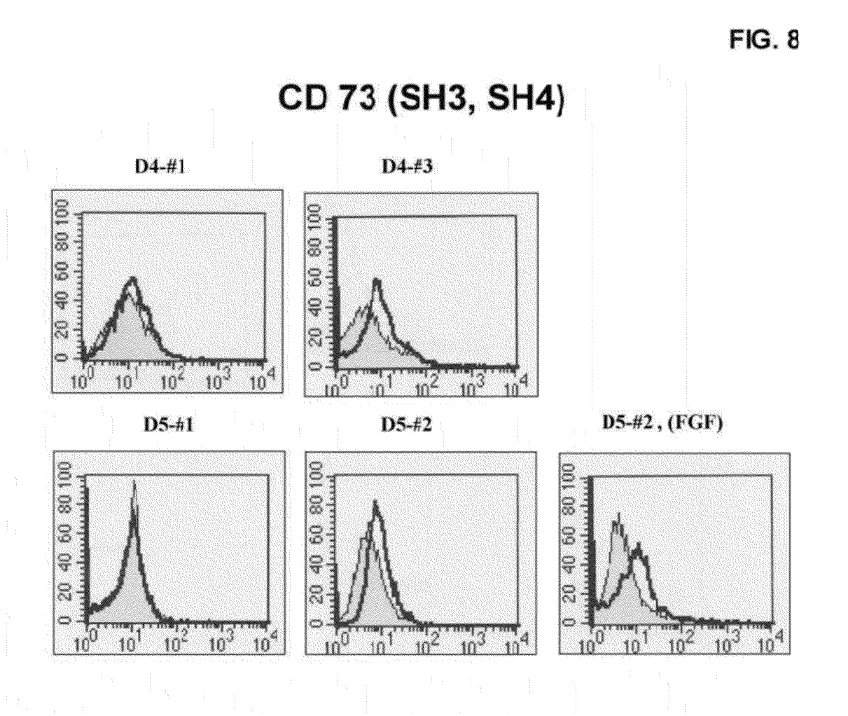

FIG. 8 shows a comparison of cell surface protein CD73 (SH3, SH4) expression. The established MLSC line D4(#1) shows a very low level of CD 73 expression and D4(#3) and D5(#2) show an intermediate level, whereas D5(#1) does not express it at all.

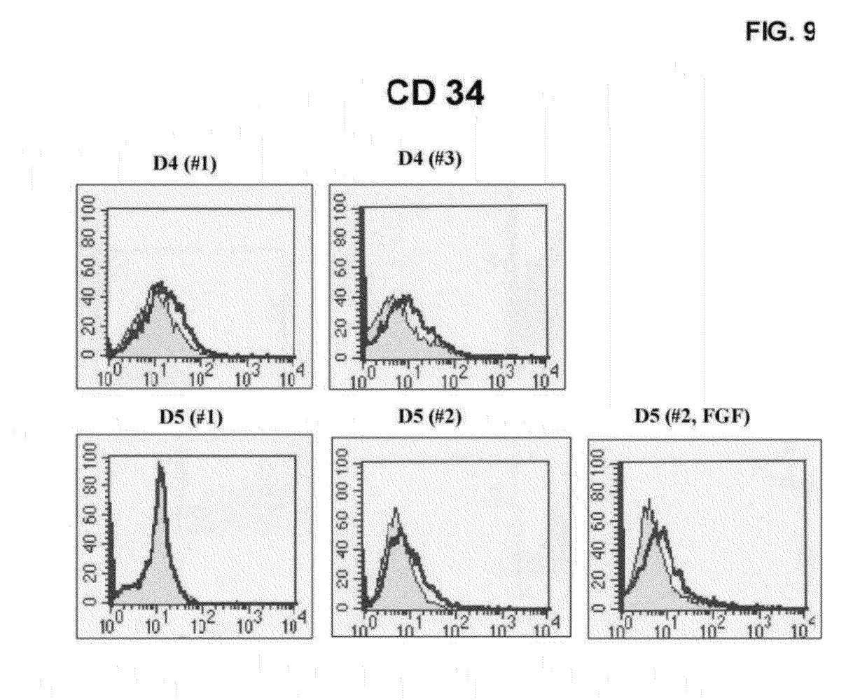

FIG. 9 shows comparison of cell surface protein CD34 expression. The established MLSC lines D4(#3), D4(#3), and D5(#2) show low level of CD34 expression, whereas D5(#1) shows no CD34 expression. The above results indicate that each stem cell line has unique cell characteristics in its differentiation capability and/or cell function.

### Example 6.3

### Multi-Lineage Differentiation of Bone Marrow Cell Lines

In order to determine the differentiation capacity of the single-cell derived bone marrow cell lines, chondrogenic, osteogenic, and adipogenic differentiation were tested by pellet-culture system and neurogenic and hepatogenic differentiation by normal cell culture in each cell-specific induction medium. All the isolated cell lines were capable of differentiating into chondrogenic, osteogenic, adipogenic, neurogenic and hepatogenic lineages (Table 2). The four isolated MLSC lines showed different level of differentiation capability. For example, D5(#2) stem cell line is capable of differentiating to chondrocyte, osteocyte, adipocyte, hepatocyte, and neural cells, whereas others have different level of differentiation capability in osteogenic, neurogenic, or hepatogenic lineage.

TABLE 2
Summary of differentiation capability of the isolated MLSC lines

| | D4(#1) | D4(#3) | D5(#1) | D5(#2) | D5(#2, FGF) |
|---|---|---|---|---|---|
| Chondrogenic | H | H | H | H | I |
| Osteogenic | N | L | I | H | H |
| Adipogenic | H | H | H | H | H |
| Neurogenic | I | H | L | H | H |
| Hepatogenic | L | L | N | H | H |

(N—negative, L—low, I—intermediate, H—high)

### Example 6.4

### Chondrogenic Differentiation of Bone Marrow Cell Lines

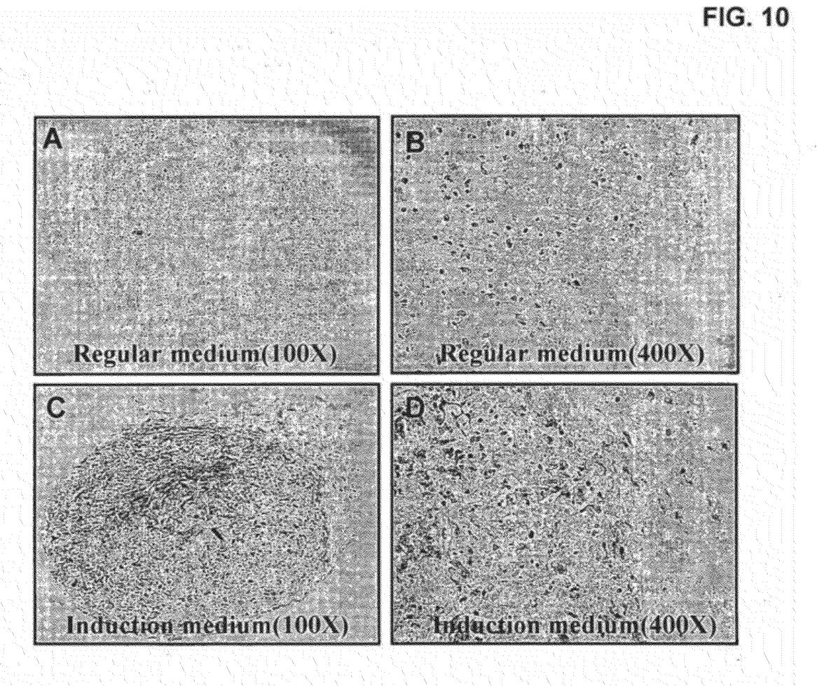

Four single-cell derived bone marrow cell lines at passage 3 or 4 were pellet-cultured in chondrogenic differentiation medium (6.25/ml insulin, transferrin, 6.25 ng/ml selenous acid, 1.25 mg/ml BSA, 5.35/ml linoleic acid, TGF-β1 10 ng/ml, and TGF-β3 10 ng/ml). Chondrogenic differentiation was achieved 14 to 21 days following treatment. Positive toludine blue histochemical stain and type II collagen-rich extracellular matrix by immunohistochemical stain was evident (FIG. 10). In contrast, the cell lines cultured in normal culture medium showed negative stain.

### Example 6.5

### Osteogenic Differentiation of Bone Marrow Cell Lines

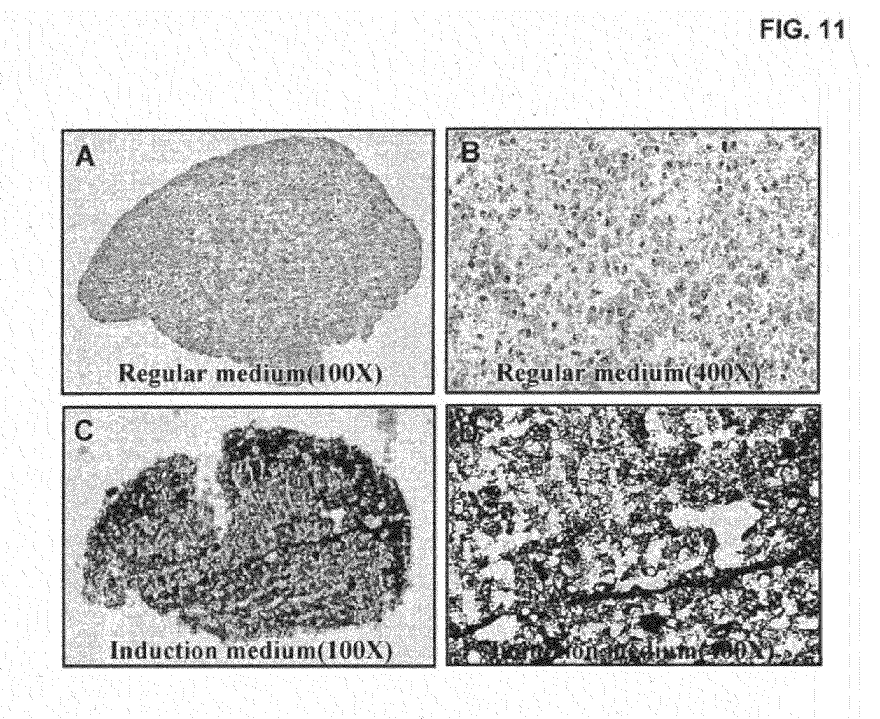

Four single-cell derived bone marrow cell lines at passage 3 or 4 were pellet-cultured in osteogenic differentiation medium (50/ml ascorbate 2-phosphate, 10⁻⁸ M dexamethasone, and 10 mM β-glycerophosphate). Osteogenic differentiation was achieved 14 to 21 days following the treatment. Postitive von Kossa staining was evident in the cells grown in osteogenic differentiation medium, while the control cells grown in normal culture was not (FIG. 11).

### Example 6.6

### Adipogenic Differentiation of Bone Marrow Cell Lines

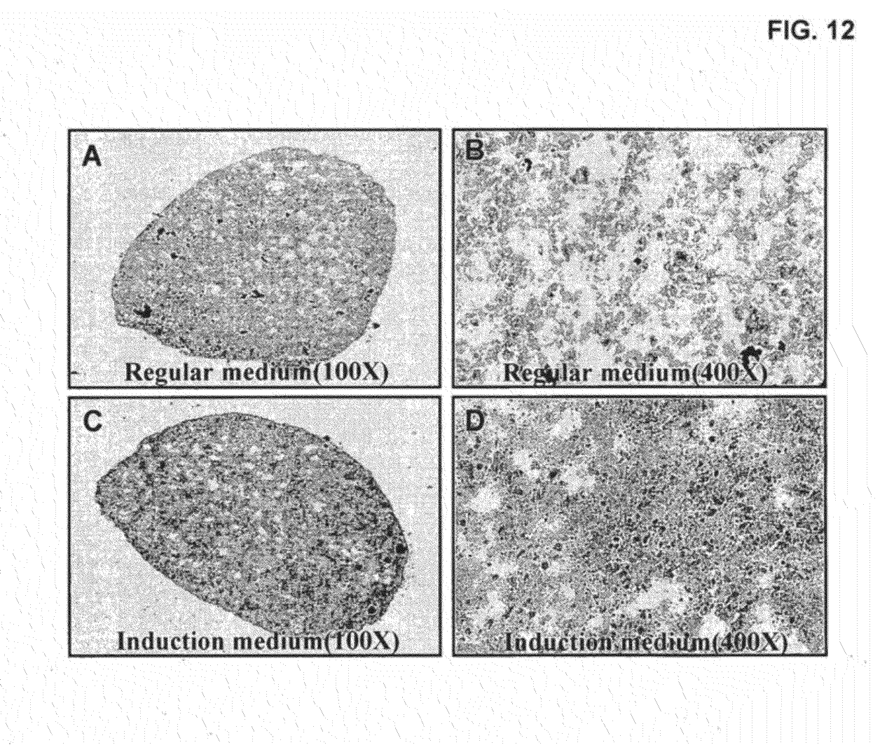

Four single-cell derived bone marrow cell lines at passage 3 or 4 were pellet-cultured in adipogenic differentiation medium (50/ml ascorbate 2-phosphate, 10⁻⁷ M dexamethasone, and 50/ml indomethacine). Adipogenic differentiation was achieved 14 to 21 days following treatment. Positive Oil red-O staining was evident in the adipogenic differentiated cells, whereas no stain was detected in the control cells grown in normal culture medium (FIG. 12).

### Example 6.7

### Neurogenic Differentiation of Bone Marrow Cell Lines

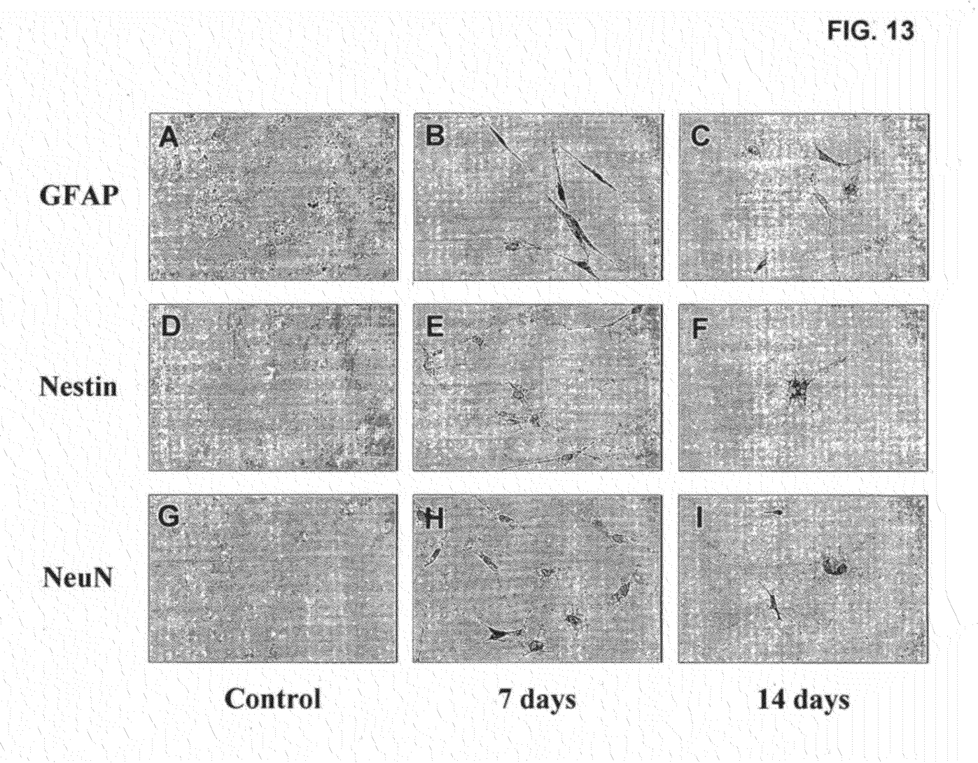

Four single-cell derived bone marrow cell lines at passage 3 or 4 were cultured in neurogenic differentiation medium (1 mM dibutyryl cyclin AMP, 0.5 mM isobutyl methylxanthine, 20 ng/ml human epidermal growth factor, 40 ng/ml basic fibroblast growth factor-8, 10 ng/ml fibroblast growth factor-8, 10 ng/ml brain-derived neurotrophic factor). Neurogenic differentiation was achieved 14 to 21 days following treatment. Positive GAFP, NueN, and Nestin staining was evident in the neurogenic differentiated cells, whereas no stain was detected in the control cells grown in normal culture medium (FIG. 13).

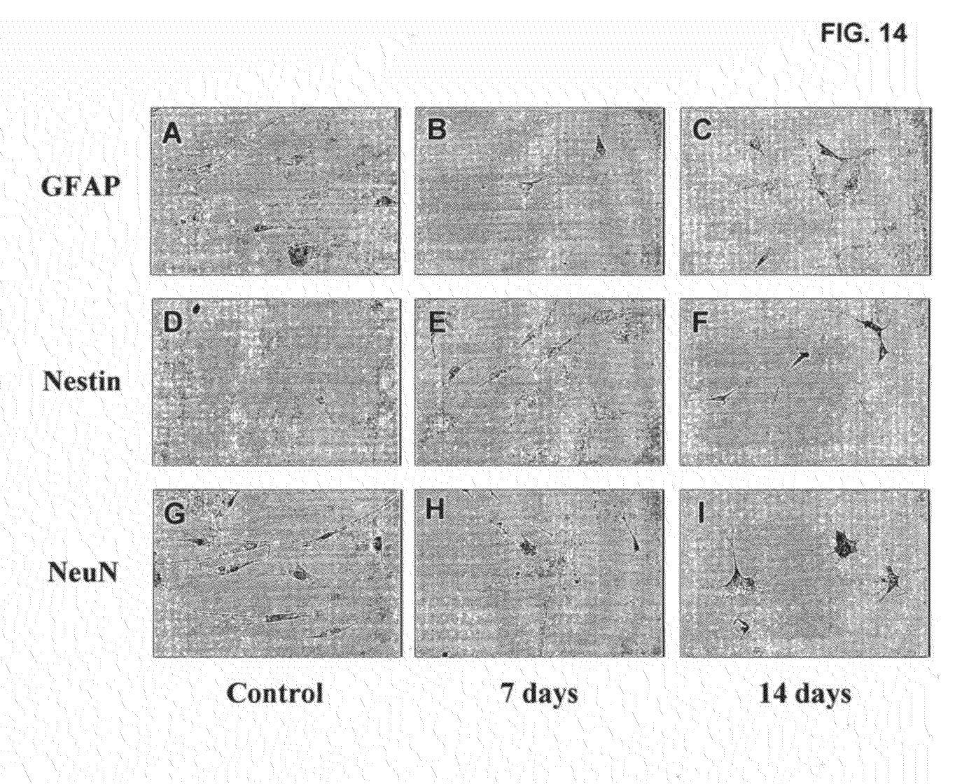

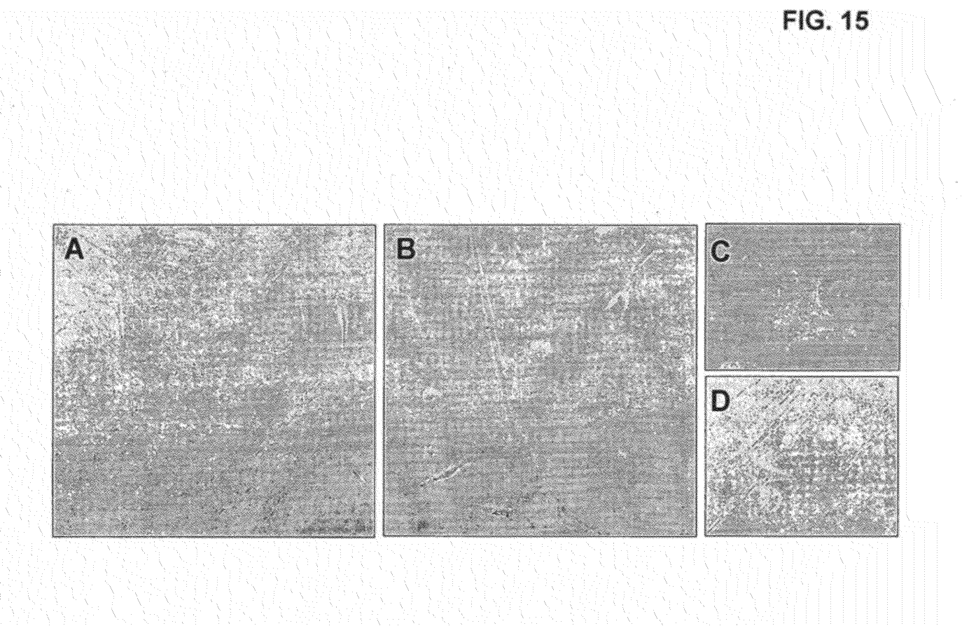

Furthermore, FIG. 14 shows neurogenic differentiation of the isolated MLSCs grown with FGF. Immunohistological stain with GFAP, Nestin, and NeuN antibodies showed that neurogenically differentiated MLSCs grown with FGF were highly positive for the stain, tested 7 and 14 days after neurogenic induction. MLSCs grown in neurogenic induction medium with FGF can also synthesize glial cell specific protein (GFAP), early and late neural cell marker proteins, Nestin and NeuN, respectively and can be differentiated into neural cells. This signifies that culturing the isolated cells with FGF did not change the neurogenic differentiation capability.

### Example 6.8

### Hepatogenic Differentiation of Bone Marrow Cell Lines

Four single-cell derived bone marrow cell lines at passage 3 or 4 were cultured in hepatogenic differentiation medium differentiation medium containing 20 mg/ml hepatocyte growth factor (R&D), 10 ng/ml oncostatin-M (R&D), 10 ng/ml epidermal growth factor (sigma), 20 ng/ml fibroblast growth factor-4 (R&D), 10 ng/ml basic-fibroblast growth factor (sigma), 50 mg/ml ITS+ premix (Becton Dickinson; 6.25 ug/ml insulin, 6.25 ug/ml transferrin, 6.25 ng/ml selenius acid, 1.25 mg/ml BSA, 5.35 mg/ml linoleic acid)), 0.1 μM ascorbate 2-phosphate (sigma), 10⁻⁸M dexamethasone (sigma). Medium was changed every 3 days (FIG. 15).

### Example 6.9

### Cartilage, Bone, Fat, Neuron, and Hepatocyte-Specific Gene Expression of Bone Marrow Cell Lines

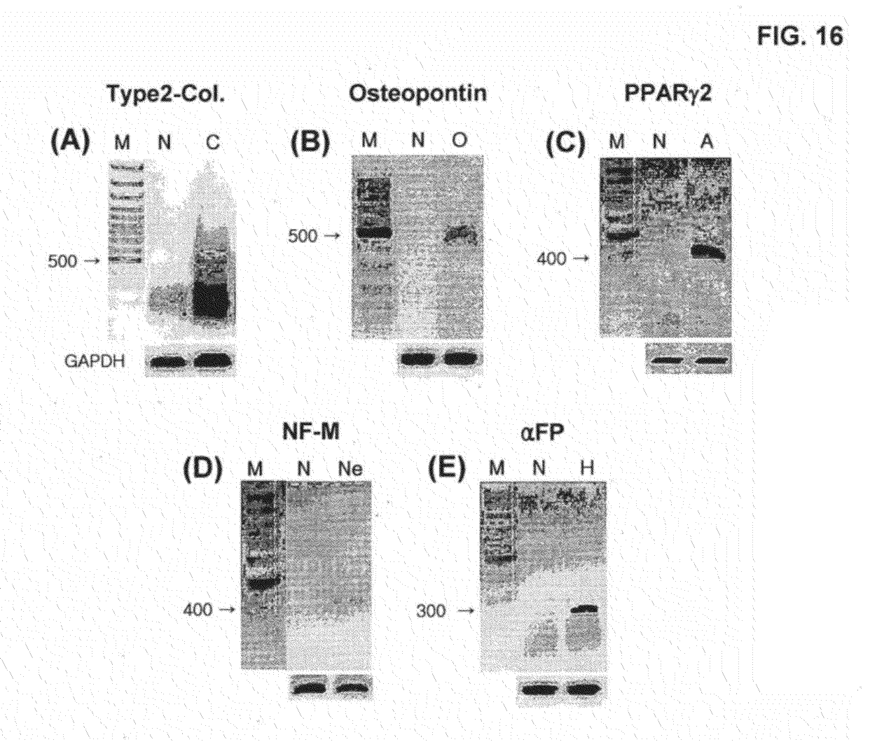

In order to measure the expression of cartilage, bone, fat, neuron and hepatocyte specific genes in the differentiated single-cell derived bone marrow cell lines, RT-PCR analysis was performed with passage 4 or 5 cells. Lineage specific gene expression of cartilage (type II collagen), bone (osteopontin), fat (PPARγ2), neuron (NF-M), and hepatocyte (αFP) were detected in the differentiated cells (FIG. 16). In contrast, these genes were not expressed in non-differentiated control cells. Expression of GAPDH was used as an internal control. These results strongly indicate that the isolated MLSCs can express cell-specific genes in each specific differentiation condition and can be differentiated into multi-lineages.

### Example 6.10

### Expression of Cytokines of Bone Marrow Cell Lines

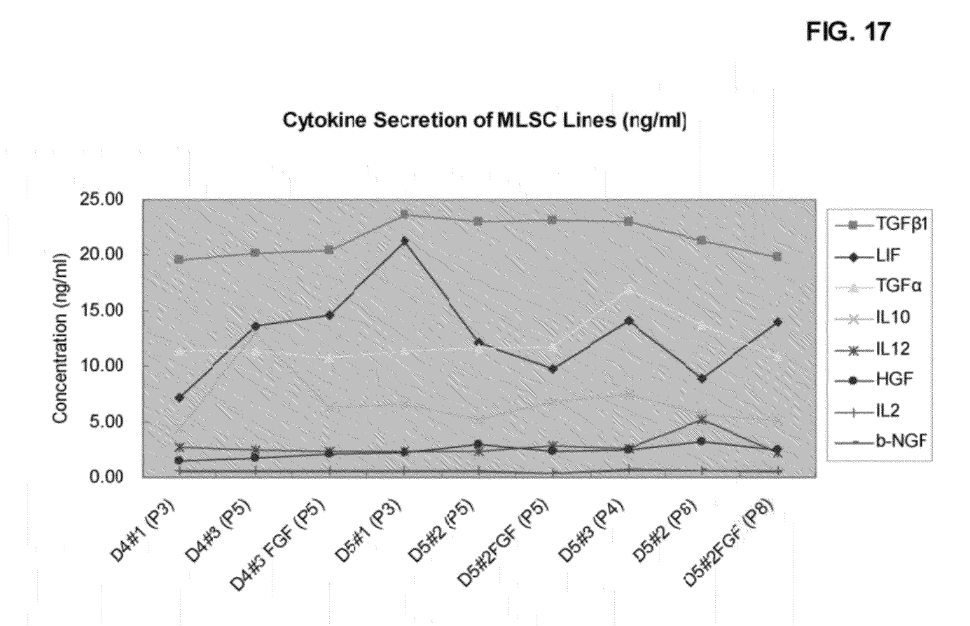

FIG. 17 shows cytokine secretion of isolated MLSC lines. Aliquots (50˜100 μl) of the MLSC culture supernatant were analyzed by ELISA using the Quantikine® Human TGF-β1, b-NGF, LIF, IL10, HGF, IL2, TGF-α and IL12. TGF-β1, LIF, TGF-β, and IL10 showed high levels of secretion, whereas the others showed low or no secretion. High level of TGF-β1 secretion by the isolated MLSCs indicates that these stem cells can play a role in suppression of T-cell activation. Also, relatively high level of other cytokines, such as LIF, TGF-β, and IL10, suggest that these cells may have immune-modulation activities.

### Example 7

### Pancreatitis Treatment Methods

### Example 7.1

### Preparation and Characterization

BM aspirates were taken from the iliac crest of a healthy female donor after obtaining informed consent (approved by Inha University Medical School Institutional Review Board). Isolation of hcMSCs was done as described above [43]. The established hcMSC line, KYJ-D2-#1, was characterized for several stem cell markers by flow cytometry. The antibodies used for the analysis were anti-CD14, anti-CD29, anti-CD31, anti-CD34, anti-CD44, anti-CD73, anti-CD90, anti-CD105, anti-CD106, anti-CD119, anti-CD133, anti-CD166, anti-CXCR-4, anti-HLA class I, anti-HLA-DR, anti-Integrin-α6, anti-PODXL, anti-Oct-4, anti-SSEA-4, and anti-Strol antibodies (BD Biosciences Pharmingen, San Diego, Calif., USA). The cells were analyzed in a FACSCalibur flow cytometer (BD Biosciences). Isotype-matched control antibodies were used as controls.

### Example 7.2

### In Vitro Adipogenic, Chondrogenic, Hepatogenic, and Osteogenic Differentiation

hcMSCs were plated in a 4-well plate at a density of 6×10⁴ cells/well. The following day, the subconfluent cells were incubated in an adipogenic medium containing 50 μg/mL ascorbic acid (Sigma, St. Louis, Mo., USA), 10⁻⁷ M dexamethasone (DEX; Sigma), 10 μg/mL insulin, 0.5 mM 1-methyl-3-isobutylxanthine (IBMX; Sigma), and 50 μg/mL indomethacine (Sigma). The cells were differentiated into adipocytes for 4-5 days. The cells were fixed with 4% formaldehyde and then stained with Oil Red O for 30 minutes followed by counterstaining with Harris hematoxylin for 10 minutes. A pellet culture system was used for chondrogenic differentiation. 2.0×10⁵ hcMSCs were put in a 15-mL conical tube and pelleted by spindown. The pellet was cultured in 500 μL serum-free chondrogenic medium (α-MEM supplemented with 10 ng/mL TGF-β1 (R&D Systems, Minneapolis, Minn., USA), 10 ng/mL TGF-β3 (R&D Systems), and 1% insulin-transferrin-selenious acid premix (BD Biosciences). The chondrogenic medium was changed every 3 days for 3 weeks. The cell pellet was embedded in an OCT compound (Sakura Finetek, Torrance, Calif., USA), frozen, sectioned into 8-mm slices, and then stained with toluidine blue. For osteogenic induction, hcMSCs seeded in a 4-well plate at a density of 6×10⁴ cells/well were cultured in an osteogenic medium (α-MEM containing 10% FBS, 50 μg/ml ascorbic acid, 10⁻⁸ M DEX, and 10 mM β-glycerophosphate). The osteogenic medium was changed every 3 days for 3 weeks. The cells were fixed using 4% paraformaldehyde, and subjected to Alizarin Red S staining.

### Example 7.3

### Immune suppression by hcMSCs

Lymph node cells were isolated from two different rat strains and 1×10⁵ cells of each strain were co-cultured in a 96-well plate. To investigate the effect of the hcMSCs on T-cell proliferation, an indicated cell number of hcMSCs were co-cultured with lymph node cells for 5 days and [³H] thymidine (1 μCi/well) were added at last 16 h of culture. The effect of hcMSCs on T-cell proliferation was determined by incorporation of [³H] thymidine. Isolated lymph node cells from rats were stimulated with different stimuli and co-cultured with hcMSCs. CD4⁺ T cells were gated and Foxp3, CD25 and Annexin V expression were analyzed by flow cytometry.

### Example 7.4

### Induction of AP

Mild-AP, edematous pancreatitis was induced by three intraperitoneal injections of cerulein (Sigma-Aldrich, St. Louis, Mo., USA) to a total dose of 100 μg/kg body weight at 2-hour intervals, with each injection containing 50% of the dose. For the severe-AP model, severe hemorrhagic pancreatitis was induced by administration of sodium taurocholate (TCA) into the bile pancreatic duct as previously described [44]. Briefly, rats were laparotomized in the midline after being anesthetized, and blunt fine catheter was introduced into the bile-pancreatic duct, and the common bile duct was clipped. A 1 ml/kg solution of 3% TCA was injected into the common bile-pancreatic duct over a 60-second period. The catheter and ligatures were then removed and the duodenal incision was closed.

Concentration of hcMSCs was based on our preliminary study (FIG. 18) and then hcMSCs (1×10⁶) was labeled with CM-1,1′-dioctadecyl-3,3,3′-tetramethylindo-carbocyanine perchloride (CM-D11) and infused on 24 hours after the last injection of cerulein and surgery by tail vein, respectively. Histological assessment was performed on pancreas tissue specimens in a blinded experiment. Interstitial edema was scored as follows: 0=absent; 1=expanded interlobular septa; 2=expanded intralobular septa; and 3=separated individual acini. Parenchymal necrosis was scored as the percentage of involvement of the examined area: 0=absent; 1=1%-10%; 2=11%-25%; and 3=>25%. Infiltration of inflammatory cells was scored as 0=absent; 1=<20 inflammatory cells per field (at 100× magnification); 2=20-50 inflammatory cells per field; and 3=>50 inflammatory cells per field.

### Example 7.5

### Analysis of hcMSCs Differentiation by Reverse Transcription-Polymerase Chain Reaction (RT-PCR)

hcMSC differentiation was verified by analyzing the gene expression of the adipogenic (aP2, peroxisome proliferator-activated receptor γ2, and lipoprotein lipase (LPL)), chondrogenic (type II collagen α1 chain (Col2A1), type X collagen α1 chain (Col10A1), and aggrecan), and osteogenic (runx2 and osteocalcin (OCN)) markers. Glyceraldehyde 3-phosphate dehydrogenase (GAPDH) was used as a loading control. PCR primer sequences used for RT-PCR analysis were as follows (F and R represent the forward and reverse primers, respectively. Annealing temperature and amplified product size are described in parenthesis): aP2 (56° C., 252 bp) (F) 5′-CATCAGTGTGAATGGGGATG-3′ (SEQ ID NO:13), (R) 5′-GTGGAAGTGACGCCTTTCAT-3′ (SEQ ID NO:14); PPARγ2 (60° C., 257 bp) (F) 5′-GACCACTCCCACTCCTTTGA-3′ (SEQ ID NO:15), (R) 5′-CGACATTCAATTGCCATGAG-3′ (SEQ ID NO:16); LPL (60° C., 717 bp) (F) 5′-TACAGGGCGGCCACAAGTTTT-3′ (SEQ ID NO:17), (R) 5′-ATGGAGAGCAAAGCCCTGCTC-3′ (SEQ ID NO:18); runx2 (58° C., 336 bp) (F) 5′-TATGAAAAACCAAGTAGCAAGGTTC-3′ (SEQ ID NO:19), (R) 5′-GTAATCTGACTCTGTCCTTGTGGAT-3′ (SEQ ID NO:20); OCN (56° C., 175 bp) (F) 5′-GTGCAGAGTCCAGCAAAGGT-3′ (SEQ ID NO:21), (R) 5′-CTAGCCAACTCGTCACAGTC-3′ (SEQ ID NO:22); Col2A1 (56° C., 498 bp) (F) 5′-TTTCCCAGGTCAAGATGGTC-3′ (SEQ ID NO:23), (R) 5′-TCACCTGGTTTTCCACCTTC-3′ (SEQ ID NO:24); Col10A1 (57° C., 703 bp) (F) 5′-GCCCAAGAGGTGCCCCTGGAATAC-3′ (SEQ ID NO:25), (R) 5′-CCTGAGAAAGAGGAGTGGACATAC-3′ (SEQ ID NO:26); aggrecan (60° C., 350 bp) (F) 5′-GCTACACCCTAAAGCCACTGCT-3′ (SEQ ID NO:27), (R) 5′-CGTAGTGCTCCTCATGGTCATC-3′ (SEQ ID NO:28); and GAPDH (56° C., 507 bp) (F) 5′-AACGGATTTGGTCGTATTGG-3′ (SEQ ID NO:29), (R) 5′-TGTGGTCATGAGTCCTTCCA-3′ (SEQ ID NO:30) (56° C., 507 bp).

To test the effect of hcMSCs on inflammatory mediators and cytokines, total RNA was extracted from the pancreas sample with Trizol reagent (Invitrogen, Carlsbad, Calif., USA) following the manufacturer's protocol. An aliquot of total RNA was reverse-transcribed and amplified using reverse transcriptase and Taq DNA polymerase (Promega, Madison, Wis., USA), respectively. The PCR product was electrophoresed on a 1.5% agarose gel. Results were recorded by an imaging system (Kodak Molecular Imaging Systems, New Haven, Conn., USA), and bands were quantified using densitometry.

### Example 7.6

### Enzyme-Linked Immunosorbent Assay (ELISA)

For the analysis of TGF-β, TNF-α, and IFN-γ in AP serum, we used rat ELISA kits (R&D Systems). The plates were coated overnight with 2 or 4 μg/mL anti-TGF-β, TNF-α, and IFN-γ capture monoclonal antibodies (in 0.1 M Na₂HPO₄ pH 9 buffer) and washed with phosphate buffered saline (PBS)-Tween 20. A biotin-labeled 1 or 2 μg/mL, anti-TGF-β, anti-TNF-α, and anti-IFN-γ detecting antibodies were used. The plates were developed using streptavidin-horseradish peroxidase (Vector, Burlingame, Calif., USA) and 2,2-azino-bis substrate (Sigma).

### Example 7.7

### Histopathology

Pancreas samples were fixed in 10% buffered formaldehyde, embedded in paraffin, and sectioned. The 8 μm-thick sections were stained with hematoxylin and eosin (H&E) for routine histology. For H&E staining, sections were stained with hematoxylin for 3 minutes, washed, and stained with 0.5% eosin for an additional 3 minutes. After a washing step with water, the slides were dehydrated in 70%, 96%, and 100% ethanol, and then in xylene. To quantify acinar cell injury, 20 randomly chosen microscopic fields were scored as previously described [44]. Briefly, edema was graded from 0-3 (0: absent; 1: focally increased between lobules; 2: diffusely increased between lobules; 3: acini disrupted and separated), inflammatory cell infiltration was graded from 0-3 (0: absent; 1: in ducts (around ductal margins); 2: in parenchyma, <50% of the lobules; 3: in parenchyma, >50% of the lobules) and acinar necrosis was graded as 0-3 (0: absent; 1: periductal necrosis, <5%; 2: focal necrosis, 5-20%; 3: diffuse parenchymal necrosis, 20-50%). TUNEL staining was performed using the ApopTag Peroxidase In Situ Apoptosis Detection Kit (Chemicon/Millipore, Billerica, Mass.) according to the manufacturer's protocol.

### Example 7.8

### Determination of Activities of Amylase, Lipase and Myeloperoxidase (MPO)

Amylase activity was assessed with a commercial kit (Bioassay, Hayward, Calif., USA) based on the use of cibachron blue-amylose as a chromogenic substrate. The soluble chromogen in 0.1 mL of serum was measured spectrophotometrically at 580 nm. The absorbance was linear to the enzyme activity. Plasma lipase activity was also determined using a commercial kit (Bioassay), following the manufacturer's instructions. The titrimetric method is based on the degradation of triolein by lipase and the consequent release of diacetylglycerol, which leads to formation of hydrogen peroxide (H₂O₂). The latter reacts with a leuco dye resulting in the formation of a chromophore that can be measured colorimetrically at 412 nm. Next, sequestration of neutrophils within the pancreas was evaluated by measuring tissue MPO activity [45]. Tissue samples were homogenized with 0.5% hexadecyltrimethyl-ammonium bromide in 50 mM phosphate buffer (pH 6.0). The suspension was subjected to four cycles of freezing and thawing, and further disrupted by sonication (40 seconds). The sample was centrifuged (10,000×g, 5 minutes, 4° C.) and MPO activity in the supernatant was assessed spectrophotometrically at 630 nm using tetramethylbenzidine as the substrate. The results were corrected in terms of protein concentration of protein, and expressed as activity per protein of the tissue (U/mg).

### Example 7.9

### Fluorescence In-Situ Hybridization (FISH) Analysis

Slides of pancreas infused with CM-DiI-labeled hcMSCs were washed and pretreated at 37° C. for 30 minutes in preheated 2° C. standard saline citrate (SSC) buffer (pH 7.0). Serial ethanol dehydration was performed (1.5 minutes each), and the slides were air-dried at room temperature. The tissue was fixed in 3:1 ethanol:acetic acid and digested with proteinase K (10 μg/mL; Sigma, St. Louis, Mo., USA) for 5 min at 37° C. Tissues were washed with water and then rinsed in 2×SSC for 3 minutes, air-dried and transferred through ice-cold 70%, 90%, and 100% ethanol, and air-dried again. Sections were denatured at 70° C. for 2 minutes in preheated 70% formamide and 2×SSC buffer (pH 7.0), and were then quenched with ice-cold 70% ethanol for 1.5 minutes. Serial ethanol dehydration was performed again. Next, a mixture of human centromere probe labeled with FITC (STAR*FISH; Cambio, Cambridge, UK) was heated at 85° C. for 10 minutes, and then applied to the sections. Coverslips were added and sealed with rubber cement for incubation overnight in a hydrated slide box at 37° C. The next day, the coverslips were carefully removed in preheated 2×SSC buffer (pH 7.0) at 37° C. The sections were washed twice in preheated 50% formamide in 2×SSC buffer for 5 minutes at 37° C. and gently washed twice in preheated 2×SSC buffer for 5 minutes. After washing, slides were mounted in a 4′,6-diamidino-2-phenylindole (DAPI) and anti-fade solution. Fluorescent staining of tissues was analyzed by confocal laser scanning microscopy (Carl Zeiss MicroImaging, Thornwood, N.Y.). Counting of FISH signal-positive nuclei was accomplished by systematically examining the FISH-stained tissue, field by field, under a ×630 or ×1000 magnification.

### Example 7.10

### Immunohistochemistry

Immunostaining was performed on 8 μm-thick sections after deparaffinization. Microwave antigen retrieval was performed in citrate buffer (pH 6.0) for 10 minutes prior to peroxidase quenching with 3% H₂O₂ in PBS for 10 minutes. Sections were then washed in water and preblocked with normal goat or rabbit serum for 10 minutes. In the primary antibody reaction, slides were incubated for 1 hour at room temperature in a 1:100 dilution of antibody. The sections were then incubated with biotinylated secondary antibodies (1:500) for 1 hour. Following a washing step with PBS, streptavidin-HRP was applied. Finally, the sections were developed with diaminobenzidine tetrahydrochloride substrate for 10 minutes, and then counterstained with hematoxylin. At least five random fields of each section were examined at a magnification of ×200 and analyzed by a computer image analysis system (Media Cybernetics, Silver Spring, Md., USA).

### Example 7.11

### Cell Proliferation and Flow Cytometry

Human peripheral blood monoclonal cells (PBMC) were isolated from two healthy donors and 1×10⁵ PBMCs of each donor were co-cultured in a flat bottom 96-well plate for a mixed lymphocyte reaction (MLR). Lymph node (LN) cells were isolated from SD and Wistar rats, and 1×10⁵ LN cells from each rat were co-cultured for MLR. To investigate the effect of the hcMSCs on T-cell proliferation, an indicated cell count of hcMSCs were co-cultured with PBMCs or LN cells for 5 days. Cell proliferation was determined by the incorporation of [³H] thymidine (1 μCi/well) after 12-16 hours of incubation. Splenocytes were isolated from SD and Wistar rats, and 1×10⁵ splenocytes from each rat were co-cultured for MLR. Next, 2×10⁵ splenocytes were stimulated with 1 μg/mL anti-CD3 (2C11) antibodies in a flat bottom 96-well plate for 3 days. The effect of hcMSCs on cell proliferation was determined by incorporation of [³H] thymidine.

### Example 7.12

### Animal Experiments

Sprague-Dawley rats weighing 180-200 g were used for the experiments. Animal care and all experimental procedures were conducted in accordance with the Guide for Animal Experiments published by the Korea Academy of Medical Science. As described in Supplementary Methods [43], two separated experimental AP models (mild- and severe-AP) were used. After induction of mild-AP, forty rats were randomly divided into four groups of ten rats; Con (n=10), Con+hcMSCs (n=10), mild-AP (n=10), and mild-AP+hcMSCs (n=10). For severe-AP study, thirty-two rats were also divided into four groups; Con (n=8), Con+hcMSCs (n=8), severe-AP (n=8), and severe-AP+hcMSCs (n=8). The rats were sacrificed by decapitation 3 days after CM-DiI-labeled hcMSCs infusion, and blood was collected and centrifuged (500×g, 25 minutes, 4° C.). Serum was used for cytokine detection as described in Supplementary Methods [44]. For evaluation of pathologic damage, pancreas sections were stained with hematoxylin and eosin (H&E) staining and were scored as described in Supplementary Methods [44]. Also, pancreas tissues were prepared for determining activities of enzymes such as amylase, lipase and myeloperoxidase (MPO) as described in Supplementary Methods [45]. To trace the injected cells in vivo, we used fluorescence in situ hybridization (FISH) for the human centromere probe labeled with FITC as described in Supplementary Methods.

Data are expressed as mean±SD. Statistical analysis was performed by using ANOVA. A P-value of <0.05 was taken to indicate statistical significance. Statistical calculations were performed using SPSS software Windows (Version 10.0; SPSS, Chicago, Ill.).

### Example 8

### Results

### Example 8.1

### Characterization of hcMSCs

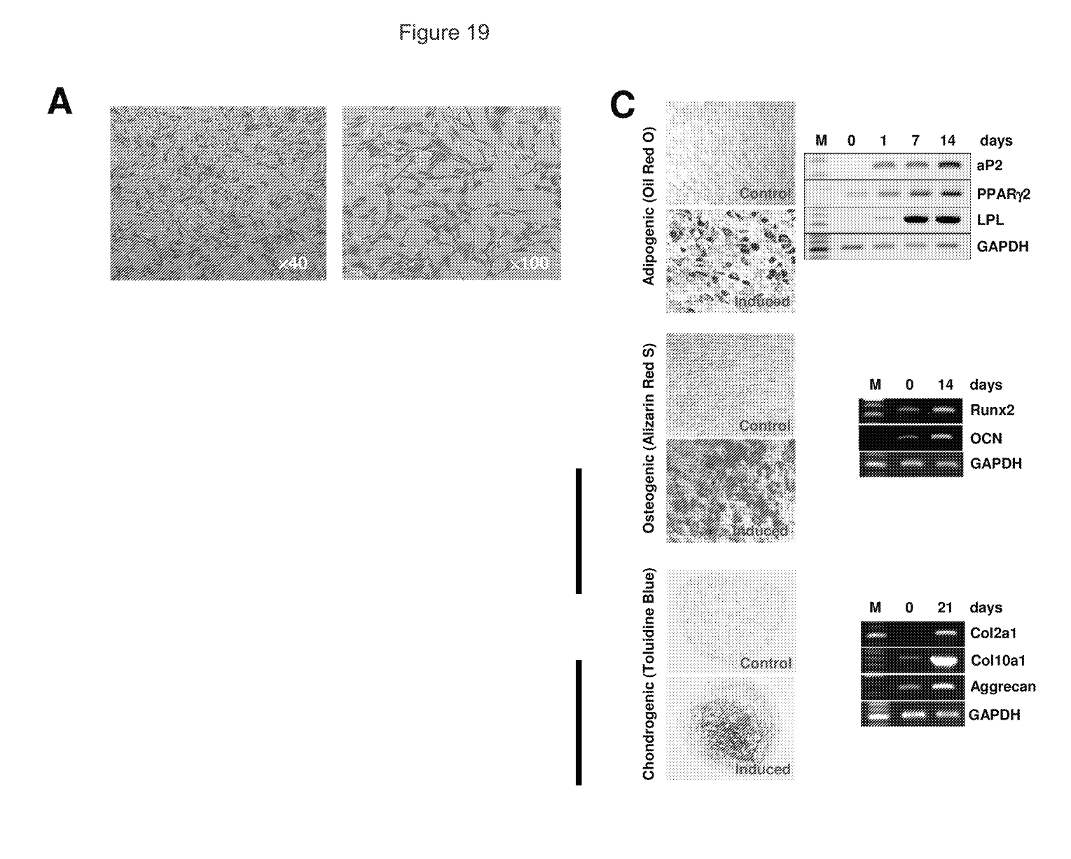

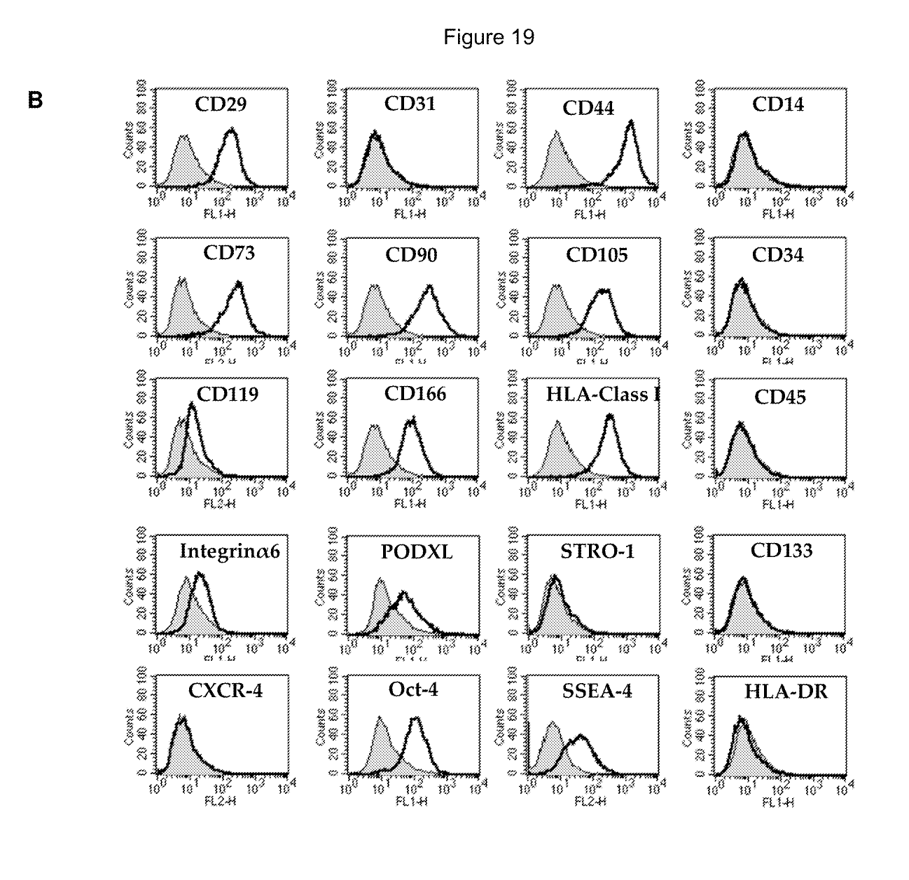

In order to obtain homogeneous adult stem cells, we isolated and established a number of hcMSCs lines from a healthy female donor through the SCM as previously described [32]. Among these, the clone, named KYJ D2-#1, was chosen and used in this study. The cells exhibited fibroblast-like shapes when cultured on plastic culture plates (FIG. 19A). They were identified by the expression of known MSC markers (FIG. 19B). They also expressed the embryonic stem cell markers Oct-4 and SSEA4 (FIG. 19B). The hcMSCs were shown to have excellent multilineage plasticity. Cell type-specific staining showed that the hcMSCs successfully differentiated into adipocytes, osteoblasts, and chondrocytes, respectively, when they were induced in vitro by adipogenic, osteogenic, and chondrogenic media. RT-PCR analysis also detected upregulation of the cell type-specific marker gene expression during differentiation (FIG. 19C). These results verified that the clonal cells isolated from human bone marrow possess MSC phenotypes.

### Example 8.2

### Histological Analysis of Pancreas after hcMSCs Infusion

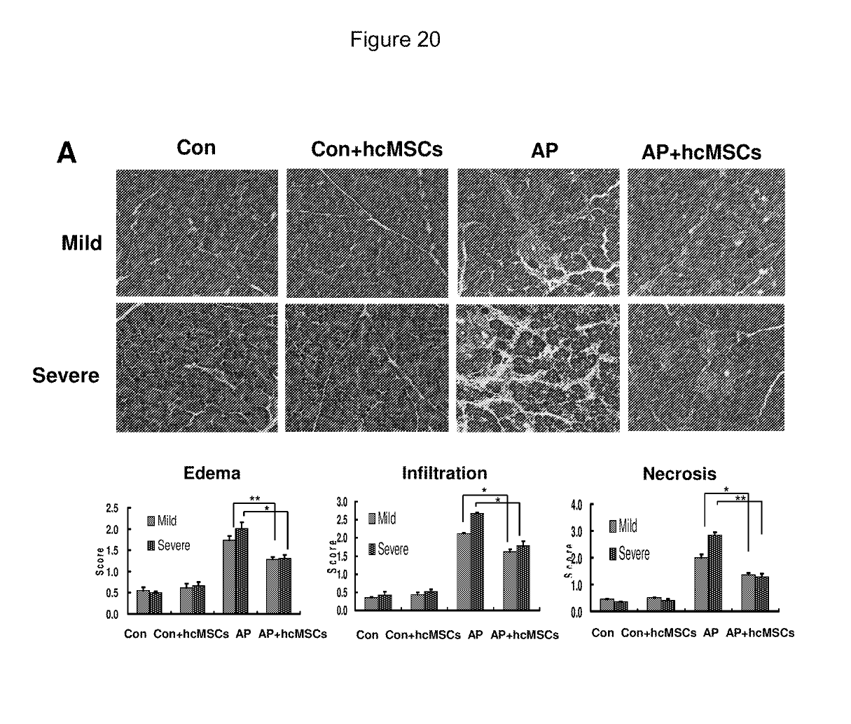

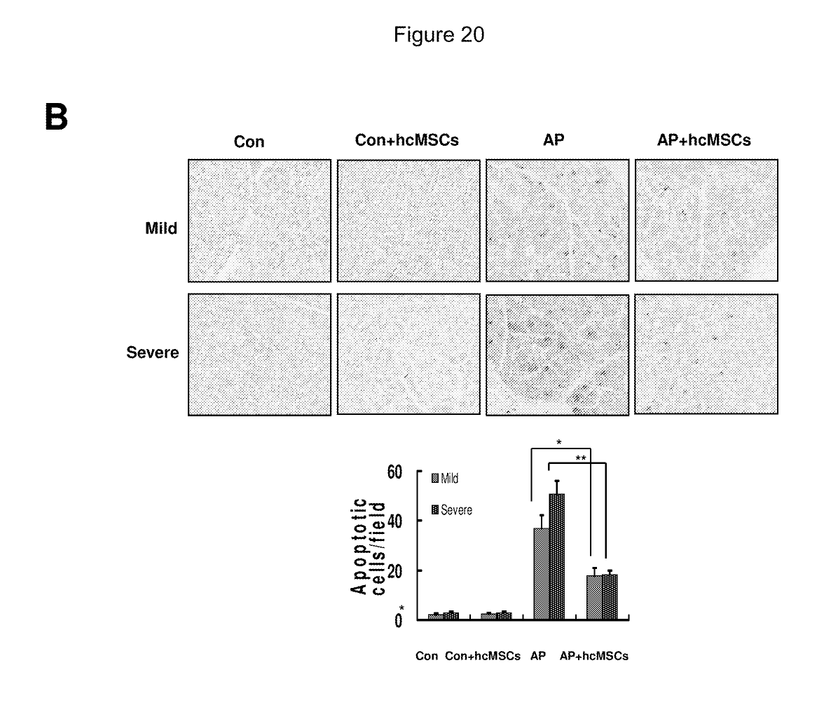

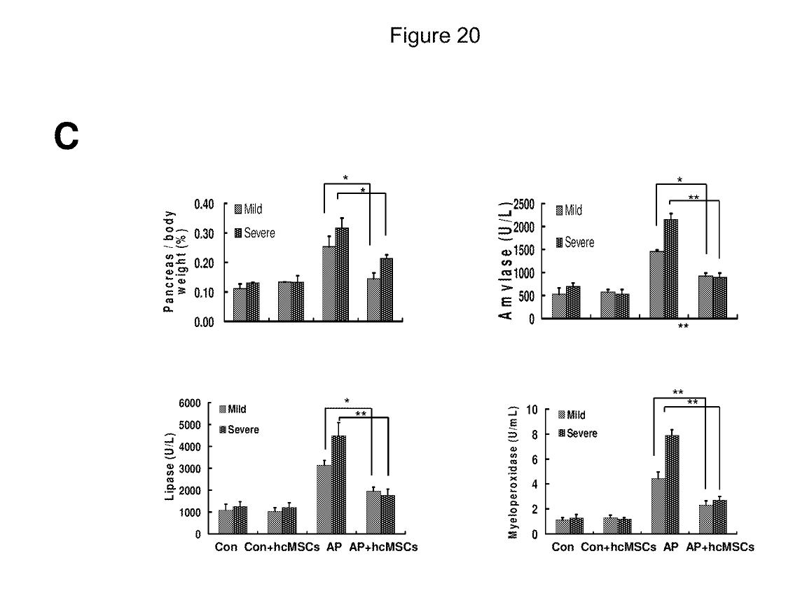

Pancreatic tissue from the mild-AP group induced by cerulein showed significant mass edema and inflammation with necrosis compared to the control and hcMSCs alone-infused groups (P<0.05). Severe-AP group induced by sodium taurocholate solution (TCA) had more profound necrosis, inflammation, and hemorrhage (data not shown), whereas edema did not show significant difference compared to mild-AP. When hcMSCs were infused to rats with mild- and severe-AP, the edema formation, inflammatory cell infiltration, and necrosis were significantly reduced in both mild- and severe-AP+hcMSCs (FIG. 20A, P<0.05 or P<0.01). The apoptotic acinar cells increased in both the mild- and severe-AP. Especially, the number of apoptotic cells was markedly higher in severe-AP than mild-AP. However, after hcMSCs infusion, the numbers of TUNEL-positive apoptotic acinar cells were reduced 1.9- and 2.5-fold in mild- and severe-AP+hcMSCs compared to mild- and severe-AP, respectively (FIG. 20B, P<0.05 or P<0.01).

### Example 8.3

### Effect of hcMSCs on the Pancreatic Markers

Pancreatitis has conspicuous indicators such as pancreatic edema and high levels of both amylase and lipase. In this study, serum amylase, lipase, and pancreatic edema were quantified to evaluate the severity of pancreatitis. As shown in FIG. 20C, serum amylase and lipase levels after hcMSCs infusion decreased about 40% and 50% in mild- and severe-AP groups, respectively. Also, the pancreas-to-body weight ratio, which reflects pancreatic edema, was significantly elevated in the mild- and severe-AP groups compared with the control group (2.1-fold and 3.2-fold, respectively, P<0.01). When hcMSCs were infused to mild- and severe-AP rats, pancreatic edema was significantly reduced (1.5-fold and 1.7-fold, respectively, P<0.05). MPO which is located in neutrophil azurophilic granules and in monocyte lysosomes, can be used to measure the extent of tissue infiltration in these cells. As expected, MPO activities of hcMSCs-infused rats were also significantly suppressed in mild- and severe-AP rats (P<0.01).

### Example 8.4

### In Vivo Tracking of hcMSCs

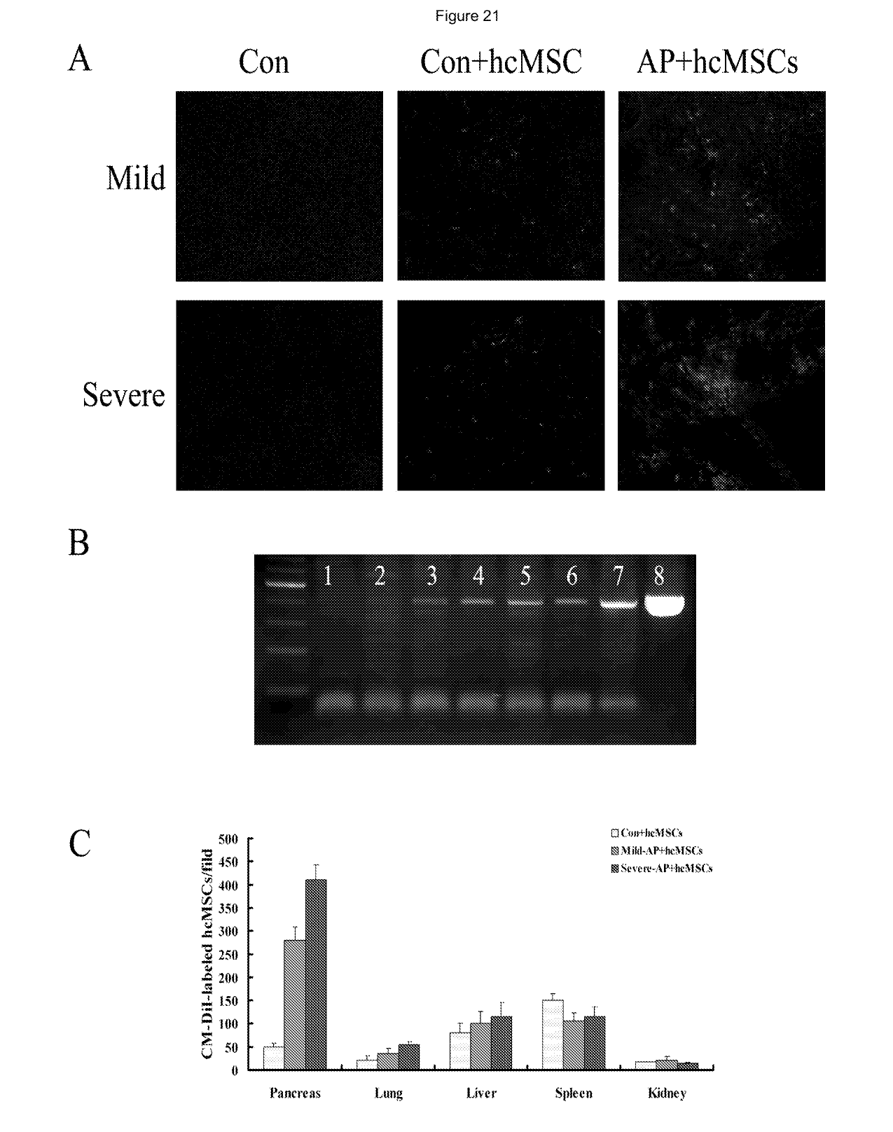

Using confocal microscopy and PCR analysis, we investigated whether hcMSCs migrate to the injured pancreas after hcMSCs infusion. CM-1,1′-dioctadecyl-3,3,3′-tetramethylindo-carbocyanine perchloride (CM-DiI) was selected as the labeling system for in vivo cell tracking of hcMSCs. hcMSCs were evenly labeled with CM-DiI, and cell labeling was achieved by optimizing dye concentration to 1 μg/L (FIG. 21A). CM-DiI labeled cells in the hcMSCs-infused group displayed red fluorescence in both mild- and severe-AP groups. Interestingly, higher number of CM-DiI labeled-hcMSCs was detected in severe-AP than in mild-AP. On the other hand, the hcMSCs alone-infused group without pancreas injury showed a smaller number of CM-DiI labeled cells than the hcMSCs-infused group with pancreas injury. Also, the presence of human cells in pancreas was confirmed by PCR for human-specific AluI sequence; the AluI was not observed in the control group, whereas it was detected in the hcMSCs alone-infused group and the mild- and severe-AP+hcMSCs groups (FIG. 21B), supporting the results from confocal microscopy analysis with CM-DiI-labeled cells. In addition, much lower number of CM-DiI labeled-hcMSCs was shown in lung, liver, spleen, kidney, compared with those in pancreas (FIG. 21C).

### Example 8.5

### Detection of hcMSCs by FISH Analysis

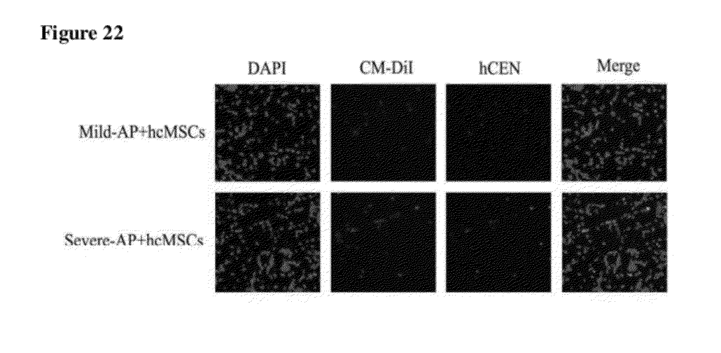

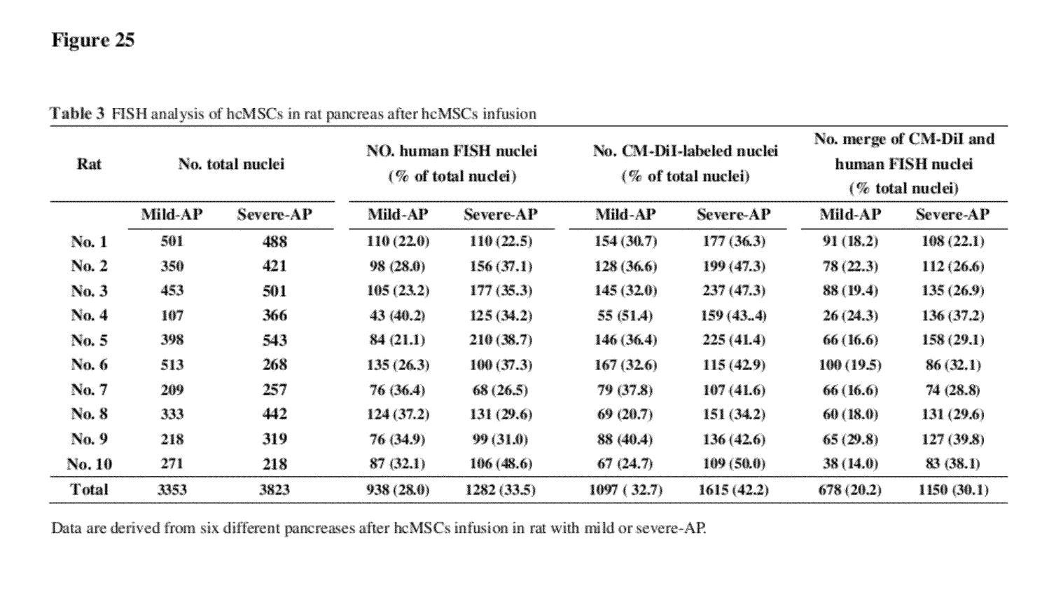

To further verify the identification of human cells, we first labeled the hcMSCs with CM-DiI and then infused them into SD rats. Three days after infusion, we analyzed the presence of human-specific chromosomal DNA by FISH, using a human chromosome centromere (green: FITC) in pancreas specimens from mild- and severe-AP rats infused with hcMSCs. After hcMSCs infusion in rats with mild- and severe-AP, in the area where hcMSCs were positively stained with CM-DiI (red), only green fluorescent dots for human chromosomes centromere (hCEN) were detected within the nuclei, identified by blue fluorescence of DAPI staining (FIG. 22). Interestingly, much more human chromosomes were observed in hcMSCs stained with CM-DiI in the severe-AP+hcMSCs group than in the mild-AP+hcMSCs group. The results from analysis of ten specimens, and ten different areas from each pancreas are shown in Table 3 (FIG. 25). A total of 938 and 1282 hybridization signals for human chromosome probes were identified in 3353 and 3823 nuclei of mild- and severe-AP groups, respectively, suggesting truncation of nuclei during sectioning, focal plane restriction, or incomplete hybridization. Human chromosome hybridization of CM-DiI-labeled hcMSCs in the severe-AP+hcMSCs group was 30% of total nuclei, which was higher than that of the mild-AP+hcMSCs group (20% of total nuclei).

### Example 8.6

### Effect of hcMSCs on Inflammation Response

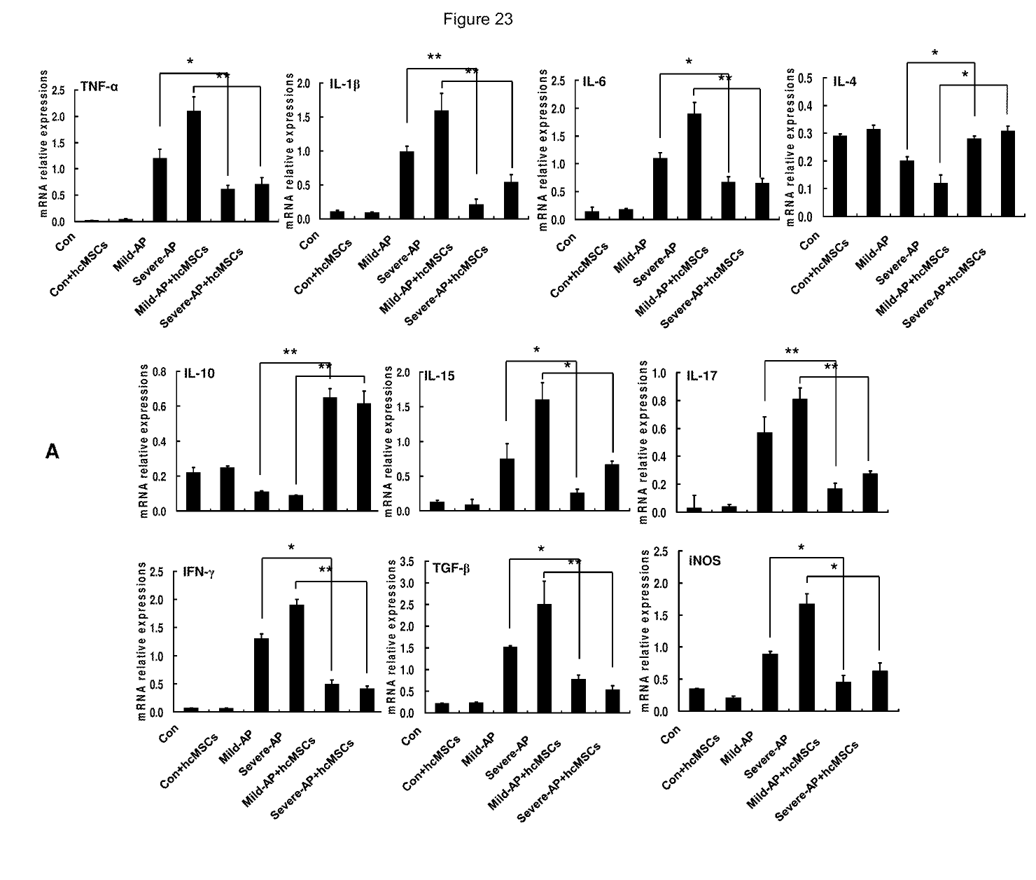

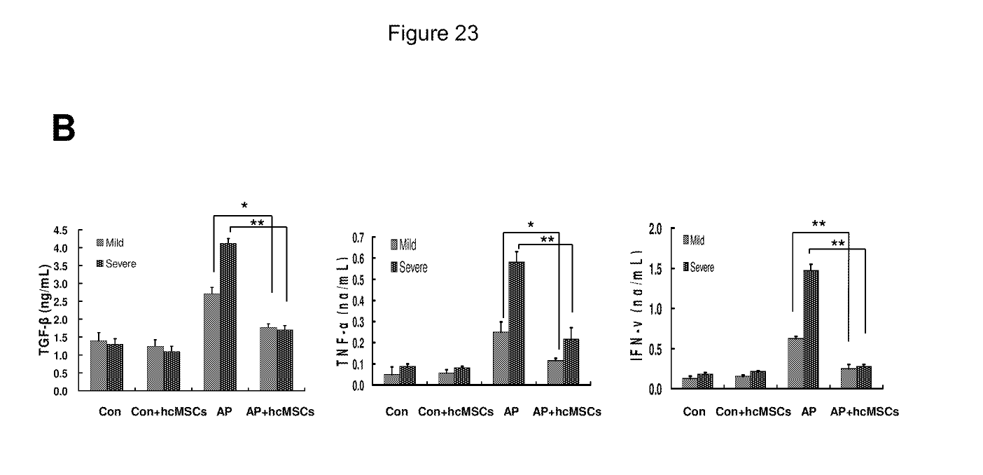

We next investigated the effects of the infusion of hcMSCs on expression and production of inflammatory mediators that are linked to AP. The mRNA of TNF-α, IFN-γ, IL-1β, IL-4, IL-6, IL-15, IL-17, iNOS and TGF-β were amplified by RT-PCR. As shown in FIG. 23A, hcMSCs significantly reduced expression level of inflammatory cytokines and mediators, including TNF-α, IFN-γ, IL-1β, IL-6, IL-15, IL-17, iNOS and TGF-β, whereas they increased expression of the anti-inflammatory cytokines such as IL-4 and IL-10 in rats with mild- and severe-AP (P<0.05 and P<0.01). In addition, the broad anti-inflammatory activity of hcMSCs in AP was accompanied by down-regulation of the systemic inflammatory response, showing decreased production of TNF-α, IFN-γ, TGF-β by ELISA (P<0.05 and P<0.01) (FIG. 23B).

### Example 8.7

### T-Cell Suppression by hcMSCs

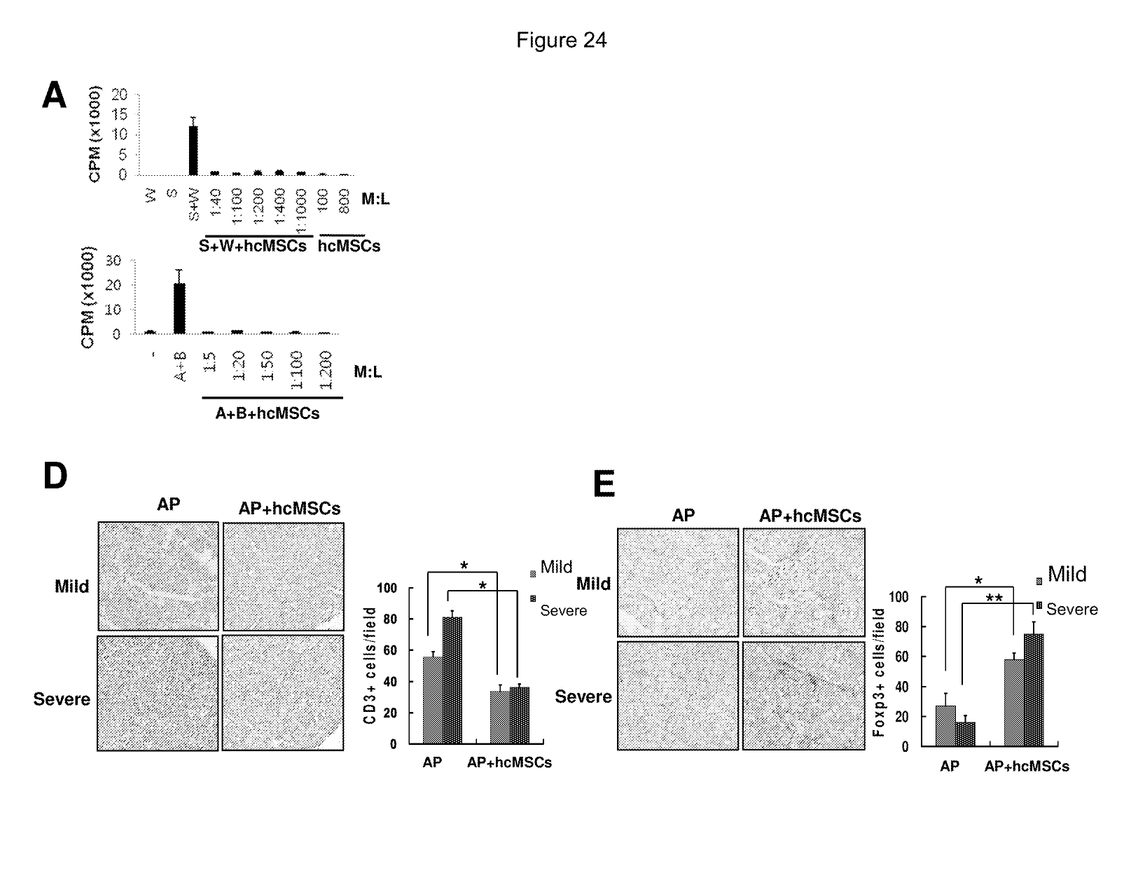

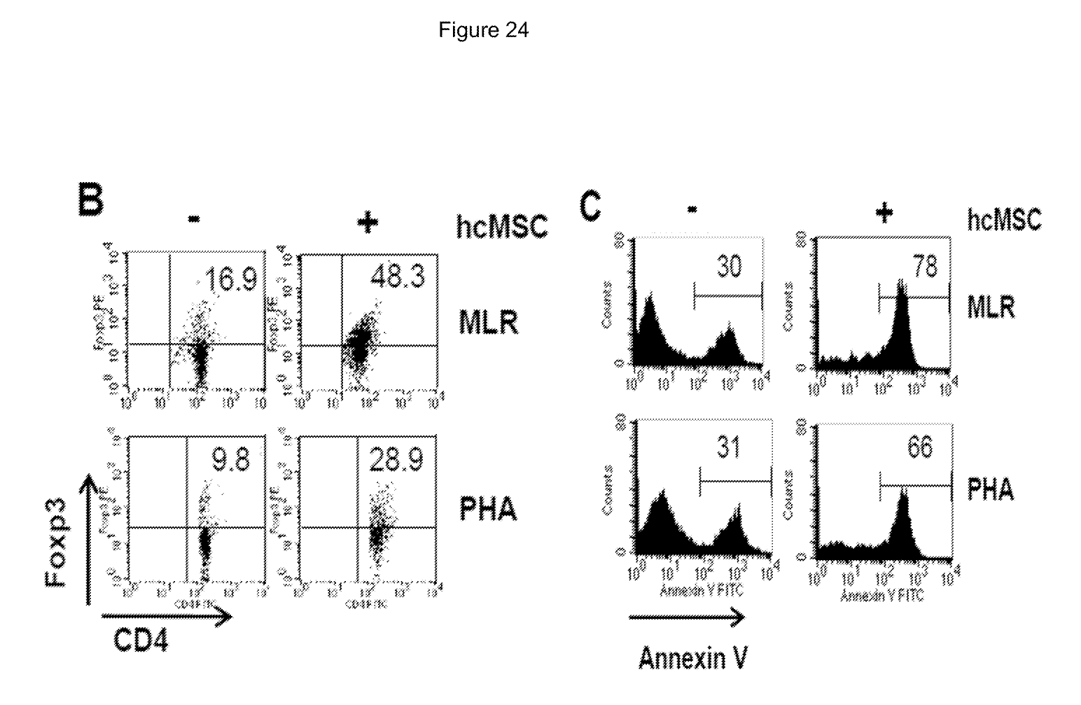

To investigate the hcMSCs effect in rats with AP, we examined whether the hcMSCs were capable of suppressing rat T-cell proliferation. 1×10⁵ lymph node cells of each strain of rats were cultured for MLR. Different number of hcMSCs (5×10³, 2×10³, 1×10³, 5×10², 2.5×10², 2×10²) were added to MLR and cultured for 5 days. As shown in FIG. 24A, the MLR was significantly suppressed by hcMSCs. A similar result was obtained when hcMSCs were co-cultured with human PBMC. We discovered that hcMSCs of passage 12-15 could effectively suppress T-cell proliferation even at a 1:1000 ratio (hcMSCs:LN cells). Recent evidences showed that MSCs can generate regulatory T cells which suppress inflammation-related diseases [44-47]. Therefore, we measured Foxp3⁺ expression in rat CD4⁺ T cells after co-culture with hcMSCs. We could find increased Foxp3⁺ expression when lymph node cells were co-cultured with hcMSCs (FIG. 24B). In addition, we observed increased Annexin V-positive CD4⁺ T cells co-cultured with hcMSCs (FIG. 24C). These results confirmed that hcMSCs-infused rats significantly decreased CD3⁺ T cell infiltration and increased expression of Foxp3⁺ regulatory T cells in pancreas tissue of rats with mild- or severe-AP (P<0.05 and P<0.01, FIGS. 24D and 24E).

All of the references cited herein are incorporated by reference in their entirety.

### REFERENCES

1. Shizuru J A, Negrin R S, Weissman I L. Hematopoietic stem and progenitor cells: Clinical and Preclinical Regeneration of the Hematolymphoid System. Annu Rev Med 2005; 56:509-538.
2. Barry F P, Murphy J M. Mesenchymal stem cells: clinical applications and biological characterization. Int J Biochem Cell Biol 2004; 36:568-584.
3. Pittenger M F, Mackay A M, Beck S C, Jaiswal R K, Douglas R, Mosca J D, Moorman M A, Simonetti D W, Craig S, Marshak D R. Multilineage potential of adult human mesenchymal stem cells. Science 1999; 284:143-147.
4. Friedenstein A J P, Petrokova K V. Osteogenesis in transplants of bone marrow cells. Journal of Embyological Experimental Morphology 1966; 16:381-390.
5. Friedenstein A J, Gorskaja J F, Kulagina N N. Fibroblast precursors in normal and irradiated mouse hematopoietic organs. Exp Hematol 1976; 4:267-274.
6. Jiang Y, Jahagirdar B N, Reinhardt R L, Schwartz R E, et al. Pluripotency of mesenchymal stem cells derived from adult marrow. Nature 2002; 418:41-49.
7. Reyes M, Verfaillie C M. Characterization of multipotent adult progenitor cells, a subpopulation of mesenchymal stem cells. Ann NY Acad Sci 2001; 938:231-233.
8. Jorgensen C, Gordeladze J, Noel D. Tissue engineering through autologous mesenchymal stem cells. Curr Opin Biotechnol 2004; 15:406-410.
9. Engineering mesenchymal stem cells for immunotherapy. Gene Ther 2003; 10:928-931.
10. Le Blanc K, Tammik C, Rosendahl K, Zetterberg E, Ringden O. HLA expression and immunologic properties of differentiated and undifferentiated mesenchymal stem cells. Exp Hematol 2003; 31:890-896.
11. Kassem M, Kristiansen M, Abdallah B M. Mesenchymal stem cells: cell biology and potential use in therapy. Basic & Clinical Pharmacology & Toxicology 2004; 95:209-214.
12. Rickard D J, Kassem M, Hefferan T E et al. Isolation and characterization of osteoblast precursor cells from human bone marrow. J Bone Miner Res 1996; 11:312-324.
13. Zohar R, Sodek J, McCulloch C A. Characterization of stromal progenitor cells enriched by flow cytometry. Blood 1997; 90:3471-3481.
14. van Vlasselaer P, Falla N, Snoeck H et al. Characterization and purification of osteogenic cells from murine bone marrow by two-color cell sorting using anti-Sca-1 monoclonal antibody and wheat germ agglutinin. Blood 1994; 84:753-763.
15. Simmons P J, Torok-Storb B. Identification of stromal cell precursors in human bone marrow by a novel monoclonal antibody, STRO-1. Blood 1991; 78:55-62.
16. Long M W, Robinson J A, Ashcraft E A et al. Regulation of human bone marrow-derived osteoprogenitor cells by osteogenic growth factors. J Clin Invest 1995; 95:881-887.
17. Waller E K, Olweus J, Lund-Johansen F et al. The “common stem cell” hypothesis reevaluated: human fetal bone marrow contains separate populations of hematopoietic and stromal progenitors. Blood 1995; 85:2422-2435.
18. Joyner C J, Bennett A, Triffitt J T. Identification and enrichment of human osteoprogenitor cells by using differentiation stage-specific mAbs. Bone 1997; 21:1-6.
19. Reyes M, Lund T, Lenvik T et al. Purification and ex vivo expansion of postnatal human marrow mesodermal progenitor cells. Blood 2001; 98:2615-2625.
20. Clark B R, Keating A. Biology of bone marrow stroma. Ann NY Acad Sci 1995; 770:70-78.
21. Phinney D G, Kopen G, Isaacson R L et al. Plastic adherent stromal cells from the bone marrow of commonly used strains of inbred mice: variations in yield, growth, and differentiation. J Cell Biochem 1999; 72:570-585.
22. Colter D C, Class R, DiGirolamo C M et al. Rapid expansion of recycling stem cells in cultures of plastic-adherent cells from human bone marrow. Proc Natl Acad Sci USA 2000; 97:3213-3218.
23. Prockop D J, Sekiya I, and Colter D C. Isolation and characterization of rapidly self-renewing stem cells from cultures of human marrow stromal cells. Cytotherapy 2001; 3(5):393-396.
24. Hung S C, Chen N J, Hsieh S L et al. Isolation and characterization of size-sieved stem cells from human bone marrow. Stem Cells 2002; 20:249-258.
25. Schwarz E J, Alexander G M, Prockop D J et al. Multipotential marrow stromal cells transduced to produce L-DOPA: engraftment in a rat model of Parkinson disease. Hum Gene Ther 1999; 10:2539-2549.
26. Schwarz E J, Reger R L, Alexander G M et al. Rat marrow stromal cells rapidly transduced with a self-inactivating retrovirus synthesize L-DOPA in vitro. Gene Ther 2001; 8:1214-1223.
27. Koc O N, Gerson S L, Cooper B W et al. Rapid hematopoietic recovery after coinfusion of autologous-blood stem cells and culture-expanded marrow mesenchymal stem cells in advanced breast cancer patients receiving high-dose chemotherapy. J Clin Oncol 2000; 18:307-316.
28. Horwitz E M, Prockop D J, Fitzpatrick L A et al. Transplantability and therapeutic effects of bone marrow-derived mesenchymal cells in children with osteogenesis imperfecta. Nat Med 1999; 5:309-313.
29. Horwitz E M, Prockop D J, Gordon P L et al. Clinical responses to bone marrow transplantation in children with severe osteogenesis imperfecta. Blood 2001; 97:1227-1231.
30. Granger J, Remick D. Acute pancreatitis: models, markers, and mediators. Shock 2005; 24 Suppl 1:45-51.
31. McKay C J, Imrie C W. The continuing challenge of early mortality in acute pancreatitis. Br J Surg 2004; 91:1243-1244.
32. Hirano T, Manabe T. A possible mechanism for gallstone pancreatitis: repeated short-term pancreaticobiliary duct obstruction with exocrine stimulation in rats. Proc Soc Exp Biol Med 1993; 202:246-252.
33. Norman J. The role of cytokines in the pathogenesis of acute pancreatitis. Am J Surg 1998; 175:76-83.
34. Lankisch P G, Lerch M M. Pharmacological prevention and treatment of acute pancreatitis: where are we now? Dig Dis 2006; 24:148-159.
35. Orlic D. Adult bone marrow stem cells regenerate myocardium in ischemic heart disease. Ann NY Acad Sci 2003; 996:152-157.
36. Kern S, Eichler H, Stoeve J, Kluter H, Bieback K. Comparative analysis of mesenchymal stem cells from bone marrow, umbilical cord blood, or adipose tissue. Stem Cells 2006; 24:1294-1301.
37. Zhao L R, Duan W M, Reyes M, Keene C D, Verfaillie C M, Low W C. Human bone marrow stem cells exhibit neural phenotypes and ameliorate neurological deficits after grafting into the ischemic brain of rats. Exp Neurol 2002; 174:11-20.
38. Augello A, Tasso R, Negrini S M, Cancedda R, Pennesi G. Cell therapy using allogeneic bone marrow mesenchymal stem cells prevents tissue damage in collagen-induced arthritis. Arthritis Rheum 2007; 56:1175-1186.
39. Togel F, Hu Z, Weiss K, Isaac J, Lange C, Westenfelder C. Administered mesenchymal stem cells protect against ischemic acute renal failure through differentiation-independent mechanisms. Am J Physiol Renal Physiol 2005; 289:F31-42.
40. Colter D C, Class R, DiGirolamo C M, Prockop D J. Rapid expansion of recycling stem cells in cultures of plastic-adherent cells from human bone marrow. Proc Natl Acad Sci USA 2000; 97:3213-3218.
41. Uccelli A, Moretta L, Pistoia V. Mesenchymal stem cells in health and disease. Nat Rev Immunol 2008; 8:726-736.
42. Song S U, Kim C S, Yoon S P, Kim S K, Lee M H, Kang J S, Choi G S, Moon S H, Choi M S, Cho Y K, Son B K. Variations of clonal marrow stem cell lines established from human bone marrow in surface epitopes, differentiation potential, gene expression, and cytokine secretion. Stem Cells Dev 2008; 17:451-461.
43. Wittel U A, Wiech T, Chakraborty S, Boss B, Lauch R, Batra S K, Hopt U T. Taurocholate-induced pancreatitis: a model of severe necrotizing pancreatitis in mice. Pancreas 2008; 36:e9-21.
44. Ren G, Zhang L, Zhao X, Xu G, Zhang Y, Roberts A I, Zhao R C, Shi Y. Mesenchymal stem cell-mediated immunosuppression occurs via concerted action of chemokines and nitric oxide. Cell Stem Cell 2008; 2:141-150.
45. Gonzalez-Rey E, Anderson P, Gonzalez M A, Rico L, Buscher D, Delgado M. Human adult stem cells derived from adipose tissue protect against experimental colitis and sepsis. Gut 2009; 58:929-939.
46. Gonzalez-Rey E, Gonzalez M A, Varela N, O'Valle F, Hernandez-Cortes P, Rico L, Buscher D, Delgado M. Human adipose-derived mesenchymal stem cells reduce inflammatory and T cell responses and induce regulatory T cells in vitro in rheumatoid arthritis. Ann Rheum Dis; 69:241-248.
47. Nemeth K, Keane-Myers A, Brown J M, Metcalfe D D, Gorham J D, Bundoc V G, Hodges M G, Jelinek I, Madala S, Karpati S, Mezey E. Bone marrow stromal cells use TGF-beta to suppress allergic responses in a mouse model of ragweed-induced asthma. Proc Natl Acad Sci USA; 107:5652-5657.
48. Gurusamy K S, Farouk M, Tweedie J H. UK guidelines for management of acute pancreatitis: is it time to change? Gut 2005; 54:1344-1345.
49. Le Blanc K, Tammik C, Rosendahl K, Zetterberg E, Ringden O. HLA expression and immunologic properties of differentiated and undifferentiated mesenchymal stem cells. Exp Hematol 2003; 31:890-896.
50. Ittrich H, Lange C, Togel F, Zander A R, Dahnke H, Westenfelder C, Adam G, Nolte-Ernsting C. In vivo magnetic resonance imaging of iron oxide-labeled, arterially-injected mesenchymal stem cells in kidneys of rats with acute ischemic kidney injury: detection and monitoring at 3T. J Magn Reson Imaging 2007; 25:1179-1191.
51. Moodley Y, Atienza D, Manuelpillai U, Samuel C S, Tchongue J, Ilancheran S, Boyd R, Trounson A. Human umbilical cord mesenchymal stem cells reduce fibrosis of bleomycin-induced lung injury. Am J Pathol 2009; 175:303-313.
52. Jung K H, Shin H P, Lee S, Lim Y J, Hwang S H, Han H, Park H K, Chung J H, Yim S V. Effect of human umbilical cord blood-derived mesenchymal stem cells in a cirrhotic rat model. Liver Int 2009; 29:898-909.
53. Laurila J P, Laatikainen L, Castellone M D, Trivedi P, Heikkila J, Hinkkanen A, Hematti P, Laukkanen M O. Human embryonic stem cell-derived mesenchymal stromal cell transplantation in a rat hind limb injury model. Cytotherapy 2009; 11:726-737.
54. Chen J, Park H C, Addabbo F, Ni J, Pelger E, Li H, Plotkin M, Goligorsky M S. Kidney-derived mesenchymal stem cells contribute to vasculogenesis, angiogenesis and endothelial repair. Kidney Int 2008; 74:879-889.
55. Weir C, Morel-Kopp M C, Gill A, Tinworth K, Ladd L, Hunyor S N, Ward C. Mesenchymal stem cells: isolation, characterisation and in vivo fluorescent dye tracking. Heart Lung Circ 2008; 17:395-403.
56. Perin E C, Silva G V, Assad J A, Vela D, Buja L M, Sousa A L, Litovsky S, Lin J, Vaughn W K, Coulter S, Fernandes M R, Willerson J T. Comparison of intracoronary and transendocardial delivery of allogeneic mesenchymal cells in a canine model of acute myocardial infarction. J Mol Cell Cardiol 2008; 44:486-495.
57. Humphreys B D, Bonventre J V. Mesenchymal stem cells in acute kidney injury. Annu Rev Med 2008; 59:311-325.
58. Tian Y, Deng Y B, Huang Y J, Wang Y. Bone marrow-derived mesenchymal stem cells decrease acute graft-versus-host disease after allogeneic hematopoietic stem cells transplantation. Immunol Invest 2008; 37:29-42.
59. Choi J B, Uchino H, Azuma K, Iwashita N, Tanaka Y, Mochizuki H, Migita M, Shimada T, Kawamori R, Watada H. Little evidence of transdifferentiation of bone marrow-derived cells into pancreatic beta cells. Diabetologia 2003; 46:1366-1374.
60. Kodama M, Tsukamoto K, Yoshida K, Aoki K, Kanegasaki S, Quinn G. Embryonic stem cell transplantation correlates with endogenous neurogenin 3 expression and pancreas regeneration in streptozotocin-injured mice. J Histochem Cytochem 2009; 57:1149-1158.
61. Marrache F, Pendyala S, Bhagat G, Betz K S, Song Z, Wang T C. Role of bone marrow-derived cells in experimental chronic pancreatitis. Gut 2008; 57:1113-1120.
62. Pereda J, Sabater L, Aparisi L, Escobar J, Sandoval J, Vina J, Lopez-Rodas G, Sastre J. Interaction between cytokines and oxidative stress in acute pancreatitis. Curr Med Chem 2006; 13:2775-2787.
63. Zyromski N, Murr M M. Evolving concepts in the pathophysiology of acute pancreatitis. Surgery 2003; 133:235-237.
64. Norman J, Franz M, Messina J, Riker A, Fabri P J, Rosemurgy A S, Gower W R, Jr. Interleukin-1 receptor antagonist decreases severity of experimental acute pancreatitis. Surgery 1995; 117:648-655.
65. Masamune A, Shimosegawa T. [Anti-cytokine therapy for severe acute pancreatitis]. Nippon Rinsho 2004; 62:2116-2121.
66. Cuzzocrea S, Mazzon E, Dugo L, Centorrino T, Ciccolo A, McDonald M C, de Sarro A, Caputi A P, Thiemermann C. Absence of endogenous interleukin-6 enhances the inflammatory response during acute pancreatitis induced by cerulein in mice. Cytokine 2002; 18:274-285.
67. Ishibashi T, Zhao H, Kawabe K, Oono T, Egashira K, Suzuki K, Nawata H, Takayanagi R, Ito T. Blocking of monocyte chemoattractant protein-1 (MCP-1) activity attenuates the severity of acute pancreatitis in rats. J Gastroenterol 2008; 43:79-85.
68. Ishida Y, Maegawa T, Kondo T, Kimura A, Iwakura Y, Nakamura S, Mukaida N. Essential involvement of IFN-gamma in Clostridium difficile toxin A-induced enteritis. J Immunol 2004; 172:3018-3025.
69. Ishida Y, Kondo T, Ohshima T, Fujiwara H, Iwakura Y, Mukaida N. A pivotal involvement of IFN-gamma in the pathogenesis of acetaminophen-induced acute liver injury. FASEB J 2002; 16:1227-1236.
70. Genovese T, Mazzon E, Di Paola R, Muia C, Crisafulli C, Malleo G, Esposito E, Cuzzocrea S. Role of peroxisome proliferator-activated receptor-alpha in acute pancreatitis induced by cerulein. Immunology 2006; 118:559-570.
71. Wildi S, Kleeff J, Mayerle J, Zimmermann A, Bottinger E P, Wakefield L, Buchler M W, Friess H, Korc M. Suppression of transforming growth factor beta signalling aborts caerulein induced pancreatitis and eliminates restricted stimulation at high caerulein concentrations. Gut 2007; 56:685-692.
72. Al-Mufti R A, Williamson R C, Mathie R T. Increased nitric oxide activity in a rat model of acute pancreatitis. Gut 1998; 43:564-570.
73. de Waal Malefyt R, Abrams J, Bennett B, Figdor C G, de Vries J E. Interleukin 10 (IL-10) inhibits cytokine synthesis by human monocytes: an autoregulatory role of IL-10 produced by monocytes. J Exp Med 1991; 174:1209-1220.
74. Christiansen J, Lafvas I, Lindqvist B. Microscopical haematuria after injection of heparin. Acta Med Scand 1970; 188:221-224.
75. Brockwell P J, MacLaren M D, Trucco E. Monte Carlo simulation of PLM-curves and collection functions. Bull Math Biophys 1970; 32:429-443.
76. Zhang C, Ge C L, Guo R X, He S G. Effect of IL-4 on altered expression of complement activation regulators in rat pancreatic cells during severe acute pancreatitis. World J Gastroenterol 2005; 11:6770-6774.
77. Laveda R, Martinez J, Munoz C, Penalva J C, Saez J, Belda G, Navarro S, Feu F, Mas A, Palazon J M, Sanchez-Paya J, Such J, Perez-Mateo M. Different profile of cytokine synthesis according to the severity of acute pancreatitis. World J Gastroenterol 2005; 11:5309-5313.
78. Mayer J, Rau B, Gansauge F, Beger H G. Inflammatory mediators in human acute pancreatitis: clinical and pathophysiological implications. Gut 2000; 47:546-552.
79. Brivet F G, Emilie D, Galanaud P. Pro- and anti-inflammatory cytokines during acute severe pancreatitis: an early and sustained response, although unpredictable of death. Parisian Study Group on Acute Pancreatitis. Crit Care Med 1999; 27:749-755.
80. Konturek P C, Jaworek J, Maniatoglou A, Bonior J, Meixner H, Konturek S J, Hahn E G. Leptin modulates the inflammatory response in acute pancreatitis. Digestion 2002; 65:149-160.
81. Chen C C, Wang S S, Lu R H, Chang F Y, Lee S D. Serum interleukin 10 and interleukin 11 in patients with acute pancreatitis. Gut 1999; 45:895-899.
82. Zhang Q, Shi S, Liu Y, Uyanne J, Shi Y, Le A D. Mesenchymal stem cells derived from human gingiva are capable of immunomodulatory functions and ameliorate inflammation-related tissue destruction in experimental colitis. J Immunol 2009; 183:7787-7798.
83. Guo J, Lin G S, Bao C Y, Hu Z M, Hu M Y. Anti-inflammation role for mesenchymal stem cells transplantation in myocardial infarction. Inflammation 2007; 30:97-104.
84. Semedo P, Palasio C G, Oliveira C D, Feitoza C Q, Goncalves G M, Cenedeze M A, Wang P M, Teixeira V P, Reis M A, Pacheco-Silva A, Camara N O. Early modulation of inflammation by mesenchymal stem cell after acute kidney injury. Int Immunopharmacol 2009; 9:677-682.
85. Gonzalez M A, Gonzalez-Rey E, Rico L, Buscher D, Delgado M. Adipose-derived mesenchymal stem cells alleviate experimental colitis by inhibiting inflammatory and autoimmune responses. Gastroenterology 2009; 136:978-989.
86. D'Alessio F R, Tsushima K, Aggarwal N R, West E E, Willett M H, Britos M F, Pipeling M R, Brower R G, Tuder R M, McDyer J F, King L S. CD4+CD25+Foxp3+ Tregs resolve experimental lung injury in mice and are present in humans with acute lung injury. J Clin Invest 2009; 119:2898-2913.
87. Pandiyan P, Zheng L, Ishihara S, Reed J, Lenardo M J. CD4+CD25+Foxp3+ regulatory T cells induce cytokine deprivation-mediated apoptosis of effector CD4+ T cells. Nat Immunol 2007; 8:1353-1362.
88. Hayashi T, Ishida Y, Kimura A, Iwakura Y, Mukaida N, Kondo T. IFN-gamma protects cerulein-induced acute pancreatitis by repressing NF-kappa B activation. J Immunol 2007; 178:7385-7394.
89. Whitcomb D C (2006) Acute pancreatitis. N Engl J Med 20:2142-2150.
90. Balthazar E J, Freeny P C, vanSonnenberg E (1994) Imaging and intervention in acute pancreatitis. Radiology 2:297-306.
91. Mitchell R M S, Byrne M F, Baillie J (2003). Pancreatitis. Lancet 361:1447-1455.
92. Bradley E L (1993) A clinically based classification-system for acute-pancreatitis—summary of the international symposium on acute-pancreatitis. Arch Surg 5:586-590.
93. Ranson J H, Rifkind K M, Roses D F, et al. (1974) Prognostic signs and the role of operative management in acute pancreatitis. Surg Gynecol Obstet 1:69-81.
94. Swaroop V S, Chari S T, Clain J E (2004) Severe acute pancreatitis. JAMA 23:2865-2868.
95. Knaus W A, Draper E A, Wagner D P, Zimmerman J E (1985) APACHE II: a severity of disease classification system. Crit Care Med 10:818-829.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of treating pancreatitis, or reducing pancreatic edema or reducing relative weight of pancreas or increasing population of acinar cells or preventing necrosis of acinar cells of a subject suffering from pancreatitis, comprising:
(i) obtaining a biological sample of bone marrow, peripheral blood, cord blood, fatty tissue sample, or cytokine-activated peripheral blood cells;
(ii) allowing the biological sample of bone marrow, peripheral blood, cord blood, fatty tissue sample, or cytokine-activated peripheral blood cells to settle in a container;
(iii) transferring supernatant which contains comparatively less dense cells in the sample compared to other cells from the container to another container in a serial manner at least two times;
(iv) isolating less dense cells from the supernatant; and
(v) administering the cells obtained in step (iv) to a subject suffering from pancreatitis, wherein the bone marrow, peripheral blood, cord blood, fatty tissue sample, or cytokine-activated peripheral blood cells do not undergo centrifugation of greater than 1,000 rpm in steps (i) to (iii).

2. The method according to claim 1, wherein the sample of cells is mixed with a growth medium that does not contain any enzyme that dissociates cells from tissues.

3. The method according to claim 1, wherein the steps (ii) and (iii) are carried out at least three times.

4. The method according to claim 1, wherein the isolated cells from the supernatant are expanded in a container.

5. The method according to claim 1, wherein the container has a flat bottom.

6. The method according to claim 1, wherein the container is coated with a cell adhesive agent.

7. The method according to claim 6, wherein the cell adhesive agent comprises a polymer of any charged amino acids.

8. The method according to claim 7, wherein the cell adhesive agent is collagen, polylysine, polyarginine, polyaspartate, polyglutamate, or a combination thereof.

9. The method according to claim 1, wherein the sample of cells is obtained from bone marrow.

10. The method according to claim 1, wherein a single colony of multi-lineage stem cells or progenitor cells is isolated.

11. The method according to claim 1, wherein the biological sample of cells is obtained prior to undergoing any centrifugation.

12. The method according to claim 1, wherein the biological sample of cells is obtained after undergoing any centrifugation.

13. The method according to claim 1, which excludes centrifugation of the sample of cells.

14. The method according to claim 1, which does not use specific antibody detection of cells.

15. The method according to claim 1, wherein the pancreatitis is mild acute pancreatitis.

16. The method according to claim 1, wherein the pancreatitis is severe acute pancreatitis.

17. The method according to claim 1, wherein the subject is treated without systemic immunosuppression.

18. The method according to claim 2, wherein the enzyme is a protease.

19. The method according to claim 1, wherein the subject is tested for the effectiveness of the treatment.

20. The method according to claim 19, wherein the subject is tested for the effectiveness of the treatment by comparing reduction of pancreatic edema or reduction of relative weight of pancreas or increase in population of acinar cells or lessening of necrosis of acinar cells in the subject, before and after treatment, or relative to healthy control tissue.


SEQUENCE LISTING (30 sequences)

SEQ ID NO: 1 - DNA, 22 bp, Artificial Sequence
  SS101-F
aagatggtcccaaaggtgctcg

SEQ ID NO: 2 - DNA, 22 bp, Artificial Sequence
  SS101-R
agcttctcctctgtctccttgc

SEQ ID NO: 3 - DNA, 23 bp, Artificial Sequence
  SS102-F
ctaggcatcacctgtgccatacc

SEQ ID NO: 4 - DNA, 24 bp, Artificial Sequence
  SS102-R
cgtgaccagttcatcagattcatc

SEQ ID NO: 5 - DNA, 21 bp, Artificial Sequence
  SS103-F
gctgttatgggtgaaactctg

SEQ ID NO: 6 - DNA, 21 bp, Artificial Sequence
  SS103-R
ataaggtggagatgcaggctc

SEQ ID NO: 7 - DNA, 22 bp, Artificial Sequence
  SS104-F
aactccctcaagattgtcagca

SEQ ID NO: 8 - DNA, 21 bp, Artificial Sequence
  SS104-R
tccaccaccctgttgcttgta

SEQ ID NO: 9 - DNA, 19 bp, Artificial Sequence
  SS105-F
cgcaaagactacctgaaga

SEQ ID NO: 10 - DNA, 23 bp, Artificial Sequence
  SS105-R
cagcgatttctatatccagagcc

SEQ ID NO: 11 - DNA, 24 bp, Artificial Sequence
  SS106-F
tgcagccaaagtgaagagggaaga

SEQ ID NO: 12 - DNA, 24 bp, Artificial Sequence
  SS106-R
catagcgagcagcccaaagaagaa

SEQ ID NO: 13 - DNA, 20 bp, Artificial Sequence
  aP2
catcagtgtgaatggggatg

SEQ ID NO: 14 - DNA, 20 bp, Artificial Sequence
  aP2
gtggaagtgacgcctttcat

SEQ ID NO: 15 - DNA, 20 bp, Artificial Sequence
  PPAR 2
gaccactcccactcctttga

SEQ ID NO: 16 - DNA, 20 bp, Artificial Sequence
  PPAR 2
cgacattcaattgccatgag

SEQ ID NO: 17 - DNA, 21 bp, Artificial Sequence
  LPL
tacagggcggccacaagtttt

SEQ ID NO: 18 - DNA, 21 bp, Artificial Sequence
  LPL
atggagagcaaagccctgctc

SEQ ID NO: 19 - DNA, 25 bp, Artificial Sequence
  runx2
tatgaaaaaccaagtagcaaggttc

SEQ ID NO: 20 - DNA, 25 bp, Artificial Sequence
  runx2
gtaatctgactctgtccttgtggat

SEQ ID NO: 21 - DNA, 20 bp, Artificial Sequence
  OCN
gtgcagagtccagcaaaggt

SEQ ID NO: 22 - DNA, 20 bp, Artificial Sequence
  OCN
ctagccaactcgtcacagtc

SEQ ID NO: 23 - DNA, 20 bp, Artificial Sequence
  Col2A1
tttcccaggtcaagatggtc

SEQ ID NO: 24 - DNA, 20 bp, Artificial Sequence
  Col2A1
tcacctggttttccaccttc

SEQ ID NO: 25 - DNA, 24 bp, Artificial Sequence
  Col10A1
gcccaagaggtgcccctggaatac

SEQ ID NO: 26 - DNA, 24 bp, Artificial Sequence
  Col10A1
cctgagaaagaggagtggacatac

SEQ ID NO: 27 - DNA, 22 bp, Artificial Sequence
  aggrecan
gctacaccctaaagccactgct

SEQ ID NO: 28 - DNA, 22 bp, Artificial Sequence
  aggrecan
cgtagtgctcctcatggtcatc

SEQ ID NO: 29 - DNA, 20 bp, Artificial Sequence
  GAPDH
aacggatttggtcgtattgg

SEQ ID NO: 30 - DNA, 20 bp, Artificial Sequence
  GAPDH
tgtggtcatgagtccttcca